(19) United States
(12) Patent Application Publication
(10) Pub. No.: US 2010/0279299 A1
(43) Pub. Date: Nov. 4, 2010

(54) Devices and Methods for Heating Biological Samples

(75) Inventors: George Maltezos, Oceanside, CA (US); Adrian Fawcett, Woodbridge, CA (US); Matthew Johnston, Woodbridge, CA (US); Xing Yang, San Diego, CA (US); David Tracy, Norwalk, CT (US)

Correspondence Address: WILSON, SONSINI, GOODRICH & ROSATI, 650 PAGE MILL ROAD, PALO ALTO, CA, 94304-1050, US

(73) Assignee: HELIXIS, INC., Carlsbad, CA (US)

(21) Appl. No.: 12/753,806

(22) Filed: Apr. 2, 2010

Related U.S. Application Data
(60) Provisional application No. 61/166,535, filed on Apr. 3, 2009.
(60) Provisional application No. 61/296,801, filed on Jan. 20, 2010.
(60) Provisional application No. 61/240,951, filed on Sep. 9, 2009.
(60) Provisional application No. 61/296,847, filed on Jan. 20, 2010.

(51) Int. Cl. C12Q 1/68; C12M 1/02; C12M 1/34; C12P 19/34

(52) U.S. Cl. 435/6; 435/305.2; 435/288.4; 435/91.2

(57) ABSTRACT

This invention provides a systems and methods for regulating temperature and heat transfer in applications in which it is desirable to maintain temperature uniformity such as thermal cycling applications. A heat block is used to rapidly transfer heat to or from a set of one or more reaction vessels.

### CROSS-REFERENCE

This application claims the benefit of U.S. Provisional Application No. 61/166,535, filed Apr. 3, 2009; U.S. Provisional Application No. 61/296,801, filed Jan. 20, 2010; U.S. Provisional Application No. 61/240,951, filed Sep. 9, 2009 and U.S. Provisional Application No. 61/296,847, filed Jan. 20, 2010, the contents of which applications are incorporated herein by reference in their entirety.

### BACKGROUND OF THE INVENTION

The advent of Polymerase Chain Reaction (PCR) since 1983 has revolutionized molecular biology through vastly extending the capability to identify, manipulate, and reproduce genetic materials such as DNA. Nowadays PCR is routinely practiced in medical and biological research laboratories for a variety of tasks, such as the detection of hereditary diseases, the identification of genetic fingerprints, the diagnosis of infectious diseases, the cloning of genes, paternity testing, and DNA computing. The method has been automated through the use of thermal stable DNA polymerases and machines capable of heating and cooling genetic samples rapidly, commonly known as thermal cyclers.

Many available thermal cyclers have some intrinsic limitations. Often, PCR reactions are carried out in a multiwell microplate, in order for a large number of samples to be used at once. A metal heating block is often used to carry out the thermal cycling of the reaction samples. A metal' heating block often has difficulty achieving substantially uniform temperatures across an entire microplate. In addition, the temperature control of a conventional thermal cycler needs to be improved to avoid undesired non-specific amplification of the target sequences.

There is a need in the art for an alternative or improved heating apparatus or thermal cycler design. A desirable apparatus allows both rapid and uniform transfer of heat to a sample to effect a more specific amplification reaction of nucleic acids.

### SUMMARY OF THE INVENTION

In some aspects, an apparatus is provided herein for heating a biological sample comprising: a heater, wherein the apparatus is configured to receive at least 16 sample vessels containing a biological sample, and wherein the at least 16 sample vessels are within +/−0.2° C. when heated by the heater to at least 48° C. In some instances, the apparatus is a thermal cycler and is configured to heat and cool the biological sample at PCR reaction temperatures. In some instances, the at least 16 sample vessels are within +/−0.2° C. during the PCR reaction cycles. In some instances, the heater is a thermoelectric device. In some instances, the at least 16 sample vessels are wells of a multiwell plate. In some instances, the multiwell plate has 16, 24, 48, 96, 384 or more wells.

In some instances, the apparatus further comprises a reservoir comprising a liquid composition and a stirrer. In some instances, the reservoir comprises wells configured to receive the sample vessel. In some instances, the wells are anchored to a bottom surface of the reservoir. In some instances, the width by length of the heater is less than that of the reservoir.

In some instances, an apparatus further comprises an optical assembly having a light source and an optical detector, wherein the optical assembly is positioned such that light from the light source is directed into the at least 16 sample vessels, and light from the at least 16 sample vessels is detected by the detector. In some instances, the optical assembly comprises a plurality of light sources, wherein each of the plurality of light sources correspond to an individual sample vessel of the at least 16 sample vessels. In some instances, the optical assembly comprises a lenslet array, wherein each lenslet corresponds to each of the plurality of light sources, to direct an excitation energy to the individual sample vessels of the at least 16 sample vessels. In some instances, the optical assembly further comprises a multifunction mirror that directs excitation energy to the at least 16 sample vessels, and wherein the multifunction mirror directs emission energy from the at least 16 sample vessels to the optical detector. In some instances, the apparatus further comprises a control assembly which controls the apparatus, the light source, and the detector. In some instances, the control assembly comprises a programmable computer programmed to automatically process samples, run multiple temperature, cycles, obtain measurements, digitize measurements into data or convert data into charts or graphs. In some instances, programmable computer is in communication with the apparatus, the light source, and the detector via an internet connection. In some instances, the programmable computer is in communication with the apparatus, the light source, and the detector via a wireless communication.

In an aspect, an apparatus is provided herein for heating a biological sample comprising: a heater; and a reservoir in thermal contact with the heater, wherein the reservoir contains a liquid composition, wherein the reservoir is configured to receive at least 16 sample vessels containing a biological sample, and wherein, the at least 16 sample vessels are within +/−0.2° C. when heated by the heater to at least 48° C. In some instances, the reservoir is sealed. In some instances, the liquid composition is stirred within the reservoir. In some instances, the liquid composition fluorinated fluid.

In some instances, the apparatus further comprises a stirring device configured to move the liquid composition within the reservoir. In some instances, the stirring device is a paddle wheel. In some instances, the stirring device is a stir bar. In some instances, the stirring device is driven by a magnetic motor.

In an aspect, an apparatus provided herein for heating a biological sample comprises: a heater; a reservoir in thermal contact with the heater, wherein the reservoir contains a liquid composition, wherein the liquid composition is a fluid that does not degrade within about 5 years if the reservoir is closed; and a stirring device configured to move the liquid composition within the reservoir, wherein the apparatus is configured to receive a sample vessel that comprises a biological sample. In some instances, the fluid does not oxidize within about 5 years. In some instances, the fluid is not a liquid metal. In some instances, the fluid is a fluorinated liquid. In some instances, the fluid does not degrade composition of the reservoir over time. In some instances, the reservoir comprises silver.

In an aspect, an apparatus for heating a biological sample comprises: a heater; a reservoir in thermal contact with the heater, wherein the reservoir contains a liquid composition, wherein the liquid composition has a vapor pressure of less than about 6000 Pa at 25° C. and a thermal conductivity of greater than about 0.05 W m⁻¹K⁻¹; and a stirring device configured to move the liquid composition within the reservoir, wherein the apparatus is configured to receive a sample vessel that comprises a biological sample. In some instances, the liquid composition has a vapor pressure of less than about 1500 Pa at 25° C. In some instances, the liquid composition has a thermal conductivity of between about 0.05 and 0.1 W m⁻¹K⁻¹ when the liquid composition is not being stirred. In some instances, the liquid composition comprises a fluorinated liquid. In some instances, the liquid composition comprises Fluorinert. In some instances, the liquid composition consists essentially of a fluorinated liquid. In some instances, the liquid composition has a boiling point of between about 95 and 200° C. In some instances, the liquid composition has a viscosity less than about 2.50 cSt at 25° C. In some instances, the liquid composition has a viscosity between about 0.70 and 2.50 cSt 25° C. In some instances, the liquid composition has a vapor pressure less than water. In some instances, the reservoir is sealed.

In an aspect, an apparatus is provided herein for heating a biological sample comprising: a heater; a reservoir in thermal contact with the heater, wherein the reservoir contains a liquid composition; a heat sink in thermal contact with the heater; and a thermal baseplate in thermal contact with the heater, wherein the thermal baseplate transfer heat from the heater to the heat sink. In some instances, the top surface of the thermal baseplate has similar dimensions to the bottom surface of heater in order to transfer heat uniformly to the heat sink. In some instances, the thermal baseplate comprises the same material as the interface of the heat sink. In some instances, the thermal baseplate comprises features to prevent the heater from moving horizontally when pressure is applied to the heater vertically.

In an aspect, a method of heating a biological sample comprises: positioning a sample holder containing a biological sample into thermal contact with an apparatus of claim 1; and heating the biological sample contained by the sample holder with the apparatus. In some instances, the method comprises performing PCR on the biological sample. In some instances, the apparatus maintains the temperature of sample when heating within ±0.2° C. In some instances, the method further comprises stirring the liquid composition within the reservoir. In some instances, the heating comprises thermally cycling the biological sample between about 50-65° C. and about 90 to 100° C. In some instances, each of the thermal cycles comprise an annealing temperature and a denaturing temperature, and wherein the annealing temperature of each amplification cycle varies by less than ±0.1° C. In some instances, each of the thermal cycles comprise an annealing temperature and a denaturing temperature, and wherein the denaturing temperature of each amplification cycle varies by less than ±0.1° C. In some instances, the sample holder is a multiwell plate and the wells of the multiwell plate contain the biological sample, wherein the biological sample is a polynucleotide sample. In some instances, the method further comprises providing reagents for carrying out PCR, and dyes for detecting the level of amplification to the wells containing the biological sample, thereby creating a reaction mixture. In some instances, the method further comprises optically measuring the dyes between or during each of a plurality of amplification cycles to determine the level of amplification.

In an aspect, a method is provided herein of heating a biological sample comprising: positioning a sample holder into thermal contact with a heater, wherein the sample holder comprises at least about 16 wells containing a biological sample and is at least 1 cm in width; and heating the biological sample within the sample holder with the heater, wherein the temperature variance between at least 2 samples of the at least about 16 wells is less than ±0.2° C. In some instances, the temperature variance is less than ±0.2° C. within 10 seconds immediately after increasing or decreasing the temperature of the biological sample more than 10° C. per second.

In an aspect, a method for making a thermal heat block comprises: forming a heat block having a reservoir; filling the reservoir with a fluid at a first temperature of at least 90° C. through an opening in the heat block; and sealing the opening when the heat block and fluid, wherein when the heat block is at a second temperature less than that of the first temperature, the pressure inside the reservoir is lower than ambient pressure. In some instances, the reservoir is substantially completely filled. In some instances, the reservoir is less than 50% filled. In some instances, the fluid is a fluorinated fluid. In some instances, the temperature of the fluid when being filled is about 100° C. or greater. In some instances, the thermal block is metallic. In some instances, the thermal block comprises wells, and wherein the bottoms of the wells are connected to the bottom of the thermal block. In some instances, the reservoir comprises a stirring element.

In an aspect, a system herein comprises: a thermal cycler comprising an internet connection; and a computer in communication with the thermal cycler. In some instances, the computer is in communication with the thermal cycler through the internet connection. In some instances, the computer is in communication with the thermal cycler through a wireless connection. In some instances, the control assembly comprises a programmable computer programmed to automatically process samples, run multiple temperature cycles, obtain measurements, digitize measurements into data and convert data into charts or graphs. In some instances, the computer comprises the control assembly.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Many features of the'invention are set forth with particularity in the appended claims. A better understanding of the features and advantages of the invention will be obtained by reference to the following detailed description that sets forth illustrative embodiments, in which many principles of the invention are utilized, and the accompanying drawings of which:

FIG. 1 illustrates a top view of a thermal assembly herein.

FIG. 2 illustrates a side view of an exemplary thermal assembly of a device herein.

FIG. 3 displays another view of an exemplary embodiment of a thermal assembly of a device herein.

FIG. 4 illustrates a top view of the thermal assembly without a compression plate.

FIG. 5 illustrates a view of the thermal assembly without a compression plate, wherein the thermal assembly comprises a heat sink, a thermal baseplate, a thermal block, and mixing motors.

FIG. 6 illustrates a view of the thermal assembly without a compression plate, wherein the thermal assembly comprises a heat sink, a thermal baseplate, a thermal block, and mixing motors.

FIGS. 7A-C illustrate an exemplary thermal block herein.

FIGS. 8A-B illustrate a top view of the thermal block and mixing motors of a thermal assembly as described herein.

FIG. 9 illustrates another view of a thermal block, heating device, and mixing motors of a thermal assembly herein.

FIGS. 10A-C illustrate exemplary stirrers of the thermal block.

FIGS. 11A-F and 12A-D show exemplary embodiments of reservoir and heaters of an apparatus herein with different examples of stirring devices.

FIG. 13 illustrates thermal non-uniformity (TNU) of sample devices (1-7) as described herein when the temperature of the thermal block is 95° C.

FIG. 14 illustrates thermal non-uniformity (TNU) of sample devices (1-7) as described herein when the temperature of the thermal block is 60° C.

### DETAILED DESCRIPTION OF THE INVENTION

Disclosed herein are devices for the controlled heating of samples such as biological samples for thermal cycling reactions. The devices herein can offer temperature uniformity and distribution that is superior to much of the current technology in the art. Temperature uniformity can be highly desirable in PCR reactions, for example, where a plurality of samples in a plurality of reaction containers must be cooled and heated simultaneously.

In addition to heating of PCR samples, the devices and methods herein can be used widely in the field of biotechnology and chemistry. Examples include but are not limited to incubations of enzymatic reactions such as restriction enzymes, biochemical assays and polymerase reactions; cell culturing and transformation; hybridization; and any treatment requiring precise temperature control. Based on the present disclosure, one of ordinary skill in the art can readily adapt the disclosed technology to various analyses of biological/chemical samples which require accurate temperature control.

### I. Apparatus and System

In embodiments described herein, the multiple temperature cycles correspond to multiple cycles of nucleic acid amplification. Nucleic acid amplification can comprise real-time PCR. For example, an apparatus or system of the invention can also be sometimes referred to as a thermal cycler.

In addition to providing thermal cycling for PCR, an apparatus herein can be used widely in the field of biotechnology and chemistry as is discussed herein. The use of a liquid composition as described can result in a more uniform heat transfer and more rapid heating and cooling cycles than solid metal heat blocks, which in an example, can lead to lower error rates by DNA polymerases. Further, error rates may be decreased during long amplifications, SNP identification and sequencing reactions, because of the enhanced thermal uniformity.

As described herein, liquid can provide better thermal contact between the heater and the sample holder, and provide more uniform heat transfer. As a result, the temperatures of the samples within a sample holder can be remarkably uniform. The uniformity of temperature can decrease non-specific hybridization and can increase the specificity (for example, signal-to-noise ratio) of amplification in PCR within individual wells as well as across multiple wells located in the same heat block (or reservoir). In another embodiment, the sample holder, alone or in combination with the apparatus, emits substantially all of a signal generated therein out through a discrete portion of the sample holder, for example, the top of the holder, whereby the emitted light can be collected by an optical assembly. In yet another embodiment a light detector detects substantially all of the light emitted from a sample holder. In certain embodiments the reservoir is highly reflective and reflects light transmitted through the walls of a transparent sample holder back into the sample holder. In this way, a greater proportion of a light signal generated inside the sample holder is emitted from a discrete portion of the sample holder, whereby it can be collected by the optical assembly. In an example, collecting light from a discrete location of the holder can eliminate the necessity of removing the holder from the heat block when performing real-time PCR. Accordingly, the apparatus herein is particularly adapted for performing PCR (polymerase chain reaction), reverse transcription PCR and real time PCR. In one embodiment an apparatus comprising a reservoir comprising a liquid composition is powered by AC or DC current. In some embodiments, the apparatus is powered by a power supply. In some embodiments, a battery powers the apparatus.

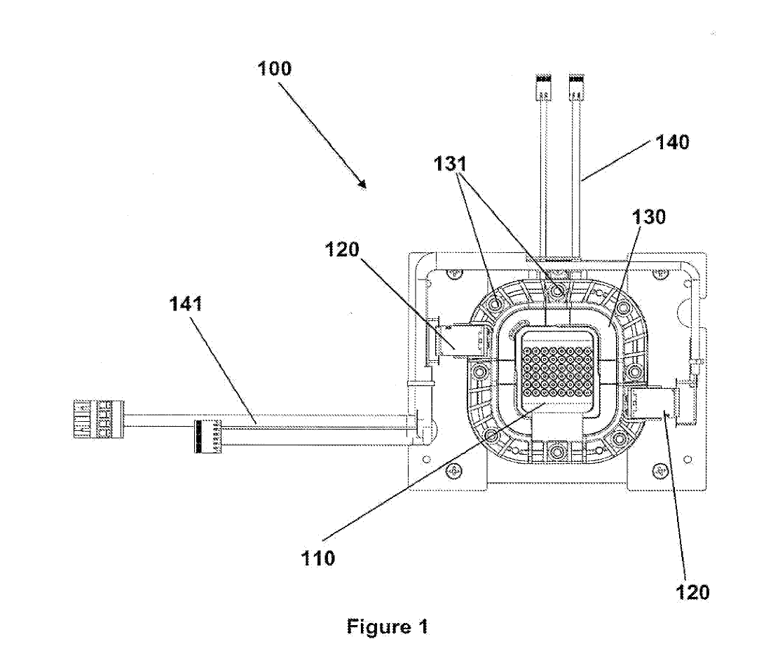

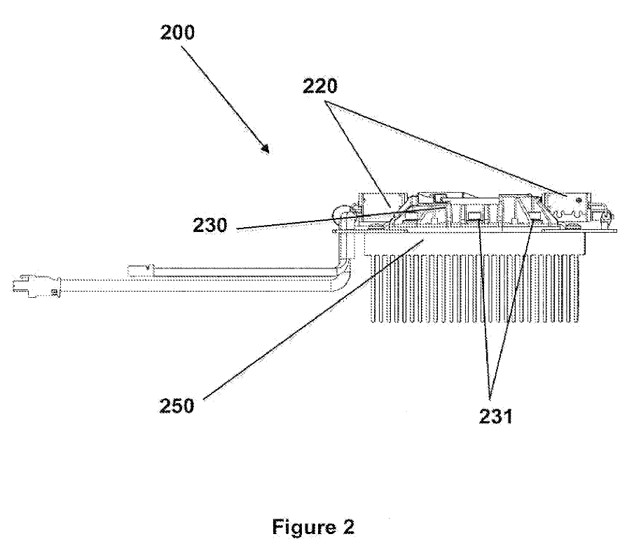

FIG. 1 illustrates a top view of a thermal assembly 100 herein. The compression plate 130 is mounted over the thermal block 110. The compression plate 130 can comprise a plastic material. In some instances, the compression plate 130 comprises glass-filled Ultem. In some instances, the compression plate 130 comprises a compliant or compressible material such as rubber, metal, polymers, ceramic, and glass. In some embodiments, the compression plate 130 is made of a material of low thermal conductivity, which, in further embodiments, minimizes thermal loss through the edge of the block 110. Also shown in FIG. 1 are compression screws 131. The screws 131 can be tightened to compress the thermal block 110 evenly against the heating device underneath the thermal block 110. In some instances, the thermal assembly 100 comprises 8 compression screws 131. In some instances, the thermal assembly 100 comprises two or more compression screws 131. In some instances, by compressing the thermal block 110 against the heating device in thermal communication, heat can be transfer from the heating device to the thermal block 110 more evenly or effectively. In some instances, the compression plate 130 provides equal or near equal force over the entire heating device from the thermal block 110. The thermal assembly 100 of the device also comprises two mixing motors 120 to drive the stirrers within the reservoir of the thermal block 110. In some instances, the thermal assembly 100 comprises mechanical motors 120 to move the stirrers within the reservoir 110. The compression plate 130 and thermal block 110 are configured to receive a microplate, for example in FIG. 1, the thermal assembly 100 is configured to receive a 48-well microplate. FIG. 1 also displays power control assemblies 141 for the mixing motors 120 and the thermal block 110. In some instances, the power control assemblies 141 are connected to a computer system to control the amount of power to the thermal block 110 and mixing motors 120. The temperature control assembly 140 for the temperature sensors for the heat sink and thermal block 110 are separated from the power control assembly 141 for the mixing motors 120 and heating device. Herein, the terms thermal block and reservoir are often used interchangeably.

FIG. 2 illustrates a side view of an exemplary thermal assembly 200 of a device herein. The thermal assembly 200 in the figure comprises a heat sink 250, a compression plate 230, compression screws 231, and mixing motors 220.

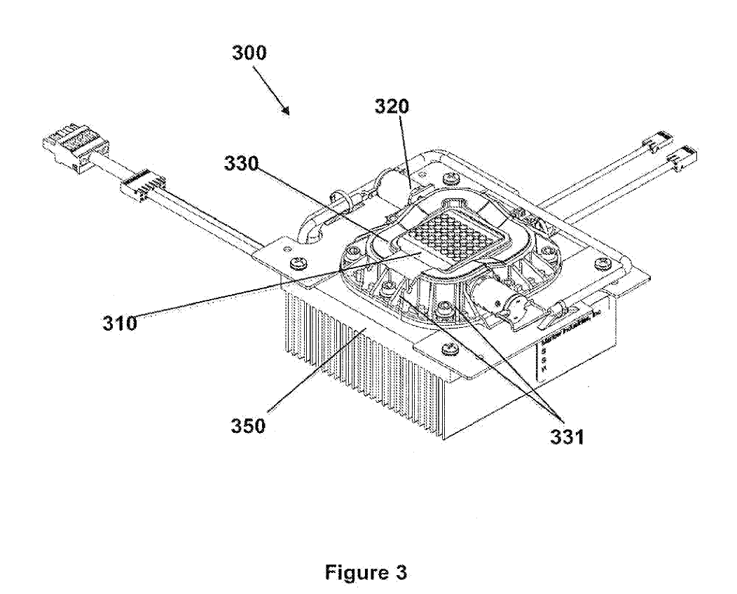

FIG. 3 displays another view of an exemplary embodiment of a thermal assembly 300 of a device herein. The thermal assembly 300 in the figure comprises a heat sink 350, a compression plate 330, compression screws 331, and mixing motors 320. Also shown is the top of the thermal block 310 comprising wells configured to receive a sample holder, such as a microplate.

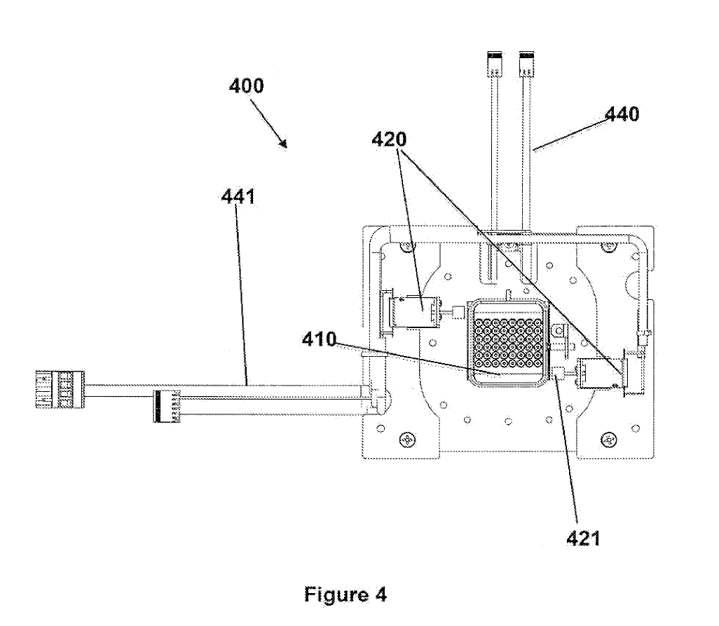

FIG. 4 illustrates a top view of the thermal assembly 400 without showing a compression plate. The figure illustrates the coupling of the mixing motors 420 with the thermal block 410. The mixing motors 420 comprise a mixing magnet 421 which couples to the magnet of the stirrers, in order to move the stirrers within the block. In some instances, as demonstrated in FIG. 4, the thermal assembly 400 comprises a temperature sensor for detecting the temperature of the thermal block 410. In some instances, the temperature sensor is in communication with the power control system, power control assembly 441, and temperature control assembly 440. The power control system can adjust the temperature output of the heating device in thermal communication with the thermal block 410 using the feedback of the temperature sensor.

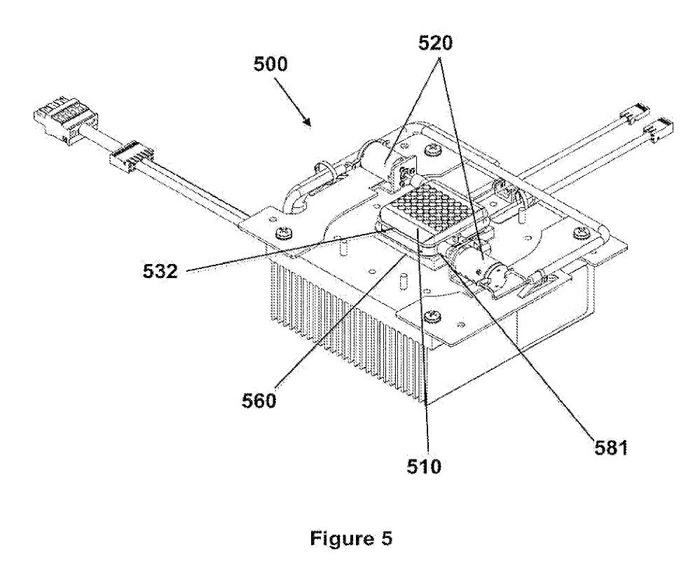

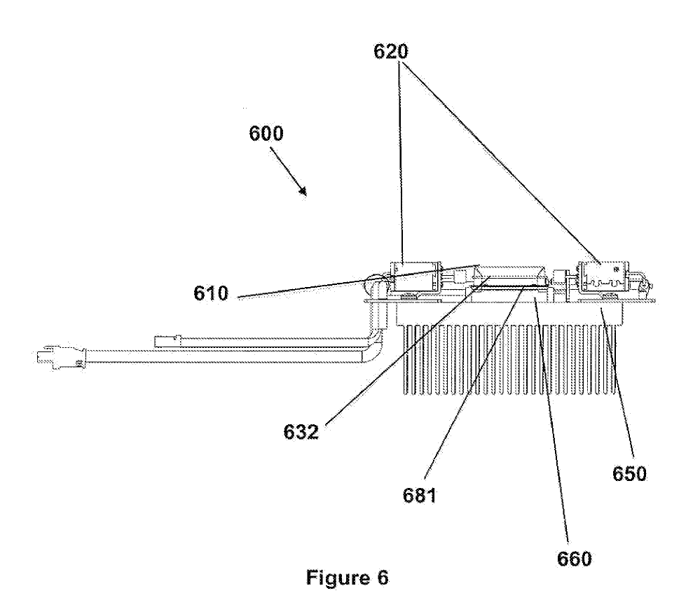

FIG. 5 demonstrates a view of the thermal assembly 500 without showing a compression plate, wherein the thermal assembly 500 comprises a heat sink, a thermal baseplate 560, a thermal block 510, and mixing motors 520. In some instances, the thermal baseplate 560 is a block that provides spacing between a heat sink and heating device. The thermal baseplate 560 is configured to conduct heat between the heating device and the heat sink in a uniform manner. The thermal baseplate 560 can have dimensions in order to transfer heat vertically into the heat sink from the heating device, as would be configured by one skilled in the art. In some instances such as the example in FIG. 5, the thermal block 510 comprises a compression gasket 532. In some instances, the compression gasket 532 is a compliant material that does not degrade at PCR reaction temperatures or below. The compression gasket 532 is configured to provide a seal between the compression plate the thermal block 510 and can prevent fluid for entering the device under the compression plate on the thermal assembly 500. Also demonstrated is a heating device gasket 581 to provide a seal between the thermal block 510 and the heating device (not shown, positioned under the thermal block 510).

FIG. 6 demonstrates a view of the thermal assembly 600 without showing a compression plate, wherein the thermal assembly 600 comprises a heat sink 650, a thermal baseplate 660, a thermal block 610, a compression gasket 632, and mixing motors 620. FIG. 6 demonstrates the heating device (as demonstrated, a Peltier device) positioned in thermal communication with the thermal block 610. As shown is a thermal baseplate 660 between the heating device and the heat sink 650, and sealed to the heating device by the heating device gasket 681. In some instances, the thermal baseplate 660 provides more efficient cooling than coupling the heating device to a heat sink 650. In some instances, the thermal baseplate 660 provides more uniform cooling than coupling the heating device to a heat sink 650. In some instances, the thermal baseplate 660 comprises a thermally conductive material. In some instances, the thermal baseplate 660 comprises the same material as the interface material of a heat sink 650. In some instances, the thermal baseplate 660 comprises features which hold the Peltier in place horizontally, so it does not move substantially when under compression. In some instances, the thermal baseplate 660 comprises features to position onto the interface of the heat sink 650. In some instances, the interface of the heat sink 650 comprises a carbon based material such as Grafoil or an equivalent material as known in the art.

### A. Reservoir

An apparatus herein can comprise a reservoir that can contain a liquid composition. The reservoir can be in thermal contact with a heater. Also, the reservoir can be in thermal contact with a sample holder, such that the reservoir provides uniform temperatures to the sample holder when the reservoir is in contact with the heater.

In some instances, the reservoir is closed. For example, the reservoir open to be filled, and once filled with a liquid composition, the reservoir can be closed. In some instances, the reservoir is closed and comprises a vacuum. For example, the reservoir can be a closed system, wherein the reservoir itself is the entire closed system. In another example, the reservoir is part of a closed system, for example, couple to a fluid loop to circulate fluid within the reservoir. In other instances, the reservoir is open and comprises at least one port.

The reservoir can comprise top, bottom, and side surfaces. In some instances, the bottom surface is closest to the heater when an apparatus is assembled. The reservoir is positioned in thermal contact with the heater. The side surfaces of the reservoir can connect the top and bottom surfaces. In some instances, a side surface has a port or opening. The reservoir can be filled through the port or opening in the side surface. The port or opening can then be closed, for example, welded or sealed, in order to create the closed system. In some instances, the port is connected to a fluid flow or pump system that can be open or closed. The top surface and the side surfaces can be a single part of the reservoir that is connected to the bottom surface to create the reservoir.

In some instances, the reservoir comprises wells configured to receive a sample holder. In many instances, the wells are in the top surface of the reservoir. The wells can be the size such that they couple closely or tightly to the sample holder to improve thermal transfer efficiency to the sample within the sample holder. In an embodiment, the wells are deeper than the wells or sample containers of the sample holder. For example, there can be a space that can be filled with solid, gas, or liquid between the bottom of the wells of the reservoir and the bottom of the sample containers of the sample holder.

The wells of the reservoir can be attached or anchored to a bottom surface of the reservoir. This can create more structural support of the reservoir. For example, if space within the reservoir is under a vacuum as compared to atmospheric pressure, wells attached to the bottom surface can act as support posts.

The reservoir can comprise a metal, polymeric, or ceramic material. In many instances, the reservoir is constructed of a material with a high thermal conductivity, such as many metals. Methods of making the apparatus and reservoir are described in more detail herein. Reservoirs may be manufactured out of any material known to be a good thermal conductor. Metal such as aluminum or copper or silver or gold may be used. In an instance, the reservoir comprises silver. Alternatively, the sample blocks may be manufactured out of composite materials such as graphite or graphite composites such as k-Core™ (k-Technology Corporation, Lancaster, Pa.). Exemplary materials for a reservoir, which sometimes can be referred to as a sample block, are also described in U.S. patent application Ser. No. 11/768,380, filed Jun. 26, 2007; U.S. patent application Ser. No. 11/433,892, filed May 12, 2006; and U.S. patent application Ser. No. 9/975,878, filed Oct. 11, 2001. In some instances, the reservoir comprises a stiff material, such as silver. In other instances, the reservoir comprises a compliant material.

In most instances, the reservoir is in thermal contact with the heater. In an example, the width by length of the heater is less than that of the reservoir. The reservoir containing the liquid composition or fluid can act as a heat spreader in order to provide temperature uniformity to the samples in the sample holder as discussed in more detail herein.

In some embodiments, the width by length of the reservoir is substantially the same (within 5%) as the width by length of the heater in which it is in thermal contact. In some embodiments, a thermal insulator may surround the reservoir and/or any elements of the apparatus such as the heater or a heat sink.

In other embodiments, the width by length of the reservoir is greater than the width by length of the heater in which it is in thermal contact. For example, some embodiments provide a heater which is in thermal contact over its top surface area with a bottom surface area of a reservoir and wherein the top surface area of the heater is 2.5%, 5%, 10%, 15%, 20%, 25%, 30%, 35%, 40%, 45%, 50%, 60%, 70%, 80%, or 90% smaller than the bottom surface area of the reservoir. In some instances, the uniformity of the temperature provided from the reservoir allows the heater to be either non-uniform, or significantly smaller in surface area than the reservoir.

In an alternative embodiment, the width by length of the reservoir is less than the width by length of the heater in which it is in thermal contact.

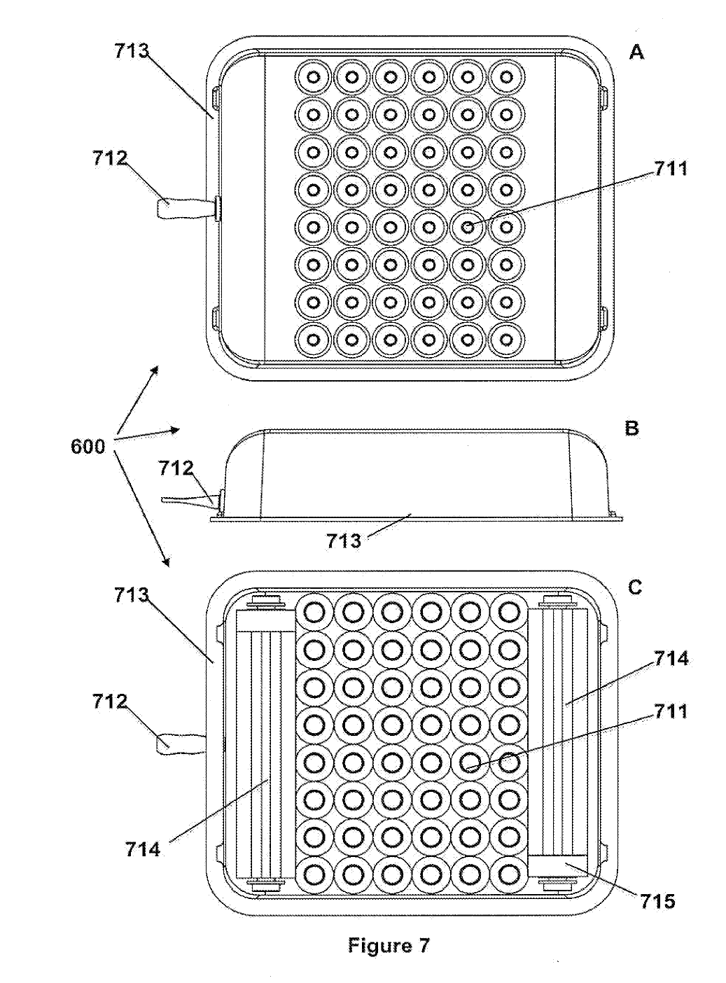

FIG. 7A illustrates an exemplary thermal block 710 herein. The block 710 comprises wells 711 configured to receive a sample, an outer cover, and a reservoir. The reservoir comprises a fluid as described herein. The reservoir can be filled and sealed through the fill inlet 712. After filling, the fill inlet 712 is closed and sealed, such that the reservoir is isolated from the environment. In some instances, the body of the block 710 comprises silver. In some instances, the thermal block 710 is configured to receive a microplate. In some instances, the thermal block 710 comprises 48 wells 711 to receive 48 wells from a sample microplate. The thermal block 710 as demonstrated in FIG. 7B comprises a flange 713 at the bottom of the thermal block 710. The flange 713 can be configured to couple to a heating device, such as a Peltier device. The flange 713 can comprise the same material as the body of the block 710. In some instances, the flange 713 is part of the bottom of the thermal block 710. In some instances, the bottom of the thermal block 710 and the flange 713 are configured to provide good thermal conductivity and/or thermal communication between the thermal block 710 and a heating device. FIG. 7C illustrates a top view of the internal composition of an exemplary thermal block 710 herein. In this example, the thermal block 710 comprises two stirrers 714. The stirrers 714 can be paddles or paddle wheels. In the example, the stirrers 714 are cylindrical and comprise paddles. The stirrers 714 also comprise magnets 715 at one end of the stirrer 714. The magnets 715 can couple with magnets of the device in order to spin the stirrers 714 and stir the fluid within the reservoir of the thermal block 710. In some instances, the stirrers 714 can rotate freely within the reservoir. In some instances, the stirrers 714 have a limited range of motion. In some instances, the stirrers 714 move the liquid within the reservoir such that it splashes the sides of the wells 711 of the thermal block 710. In this example, the thermal block 710 comprises two stirrers 714. In some instances, the thermal block 710 comprises one stirrer 714. In some instances, the thermal block 710 comprises no stirrers. In some instances, the thermal block 710 comprises 3, 4, 5, 6, 7, 8, 9, 10 or more stirrers 714.

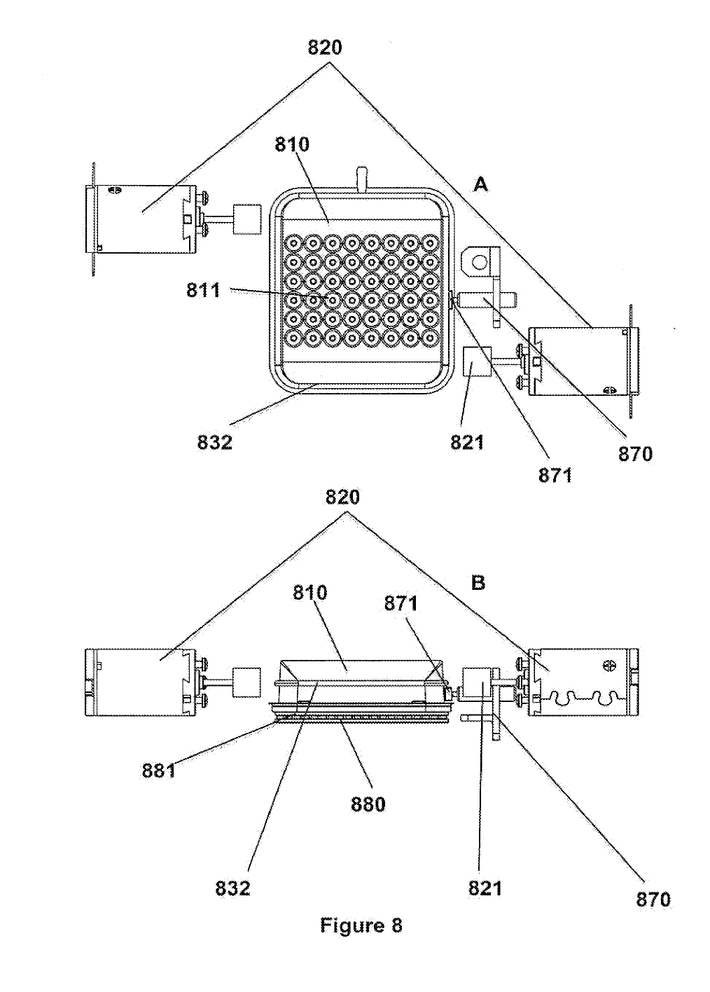

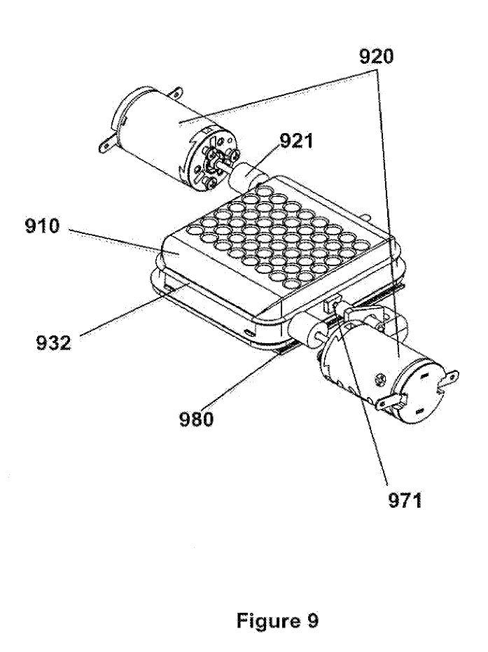

FIG. 8A illustrates a top view of the thermal block 810 with wells 811 and mixing motors 820 with the mixing magnets 821 of a thermal assembly as described herein. The thermal block 810 comprises a compression gasket 832 as described herein to prevent fluid from leaking underneath the compression gasket 832. Also shown is a temperature sensor 871 connected to a temperature sensor assembly 870, as described herein, to detect the temperature of the thermal block 810. FIG. 8B illustrates a side of a thermal block 810, heating device 880, and mixing motors 820 of a thermal assembly herein. The heating device in the example of FIG. 8B is a Peltier device. In some instances, the Peltier device is a sectioned, or diced, Peltier device. FIG. 8B also demonstrates the flange 813 of a thermal block 810 as well as the heating device gasket 881. FIG. 9 illustrates another view of a thermal block 910, a heating device 980, a block temperature sensor 971, a compression gasket 932, and mixing motors 920 with mixing magnets 921 to move stirrers of a thermal assembly herein.

### B. Liquid Composition

In an embodiment, the liquid composition has a vapor pressure less than 5620 Pa at 25° C. In an embodiment, the liquid composition has a thermal conductivity of greater than 0.05 W m⁻¹K⁻¹. In yet another embodiment, the liquid composition is a fluid that does not oxidize over time. In an embodiment, the liquid composition does not thermal degrade at PCR reaction temperatures or below. In an embodiment, the liquid composition does not chemically degrade. In an embodiment, the liquid composition does not distill over time at PCR reaction temperatures or below. For example, the fluid can be a fluorinated fluid as described herein. In some embodiments, the fluid is an oil-based fluid. In some embodiments, the fluid is a silicone oil, mineral oils, synthetic oils, naturally-occurring oils, and petrochemical oils.

The reservoir can contain a liquid composition, wherein the liquid composition has a greater Mouromsteff number in the system that provides uniform temperature distribution throughout the reservoir. A Mouromsteff number of a fluid can describe the heat transfer capability of fluid. For single phase forced convection, the Mouromsteff number (Mo) takes the form (1):

$$Mo = \frac{{ρ}^{a} {k}^{b} {c}_{p}^{d}}{{μ}^{e}} \qquad ( 1 )$$

where ρ, k, cₚ and μ represent the density, thermal conductivity, specific heat (at constant pressure), and dynamic viscosity of the fluid. The exponents a, b, d, and e take on values appropriate to the heat transfer mode of interest and the corresponding heat transfer correlation. It should be noted that the Mouromsteff number, unlike the more familiar Reynolds (ρ·V·D/μ), Nusselt (h·D/k), and Prandtl (μ·cₚ/k) numbers, is not dimensionless. The significance of the Mouromsteff number lies in the fact that for flow over or through a given geometry at a specified velocity the liquid with the largest Mouromsteff number will provide the highest heat transfer rate.

The Mouromsteff number for a given mode of heat transfer may be obtained by taking the corresponding heat transfer correlation and separating out the thermophysical property variables as a group. For fully developed, internal laminar flow, Nusselt number (h·D/k) is a constant so that the only fluid property affecting the heat transfer coefficient, h, is the thermal conductivity of the fluid. So, in this case, the Mouromsteff number is the thermal conductivity of the fluid. The heat transfer rate relative to that of water for each of the liquids is simply obtained by calculating the ratio of the Mouromsteff number for each liquid to that of water, which in this case is simply the ratio of the thermal conductivities or (2):

$$\frac{{Mo}_{fluid}}{{Mo}_{water}} = \frac{{k}_{fluid}}{{k}_{water}} \qquad ( 2 )$$

For internal turbulent flow the heat transfer rate is dependent not only upon k, but also the other thermophysical properties of the fluid. In this case the Mouromsteff number is given by (3):

$$Mo = \frac{{ρ}^{0.8} {k}^{0.67} {c}_{p}^{0.33}}{{μ}^{0.47}} \qquad ( 3 )$$

As before the heat transfer rates relative to that of water may be estimated by calculating the ratio of the Mouromsteff number for each fluid to that of water. The Mouromsteff number provides a useful reference with which to compare the heat transfer capability of liquid compositions.

In some instances, the fluid or liquid composition within the reservoir circulates with some ease when stirred and allows for efficient convection when transferring heat to a surrounding object. In some instances, the fluid has a viscosity that is lower than an oil.

A liquid composition as described herein can comprise a fluorinated liquid. In some instances, a fluorinated liquid is a perfluorinated liquid. In an example, the fluorinated liquid is Fluorinert™ (3M). In an embodiment, a liquid composition herein consists essentially of Fluorinert. Fluorinated liquids are a family of clear, colorless, odorless fluids having a viscosity similar to water but can have an approximately 75% greater density. Fluorinated liquids products are thermally and chemically stable, and are compatible with sensitive materials, including metals, plastics and elastomers, non-flammable and mostly non-toxic. A flourinated liquid can have a completely saturated hydrocarbon chain.

The dielectric strength of perfluorinated liquids is high, for example, in excess of 35,000 volts across a 0.1 inch gap. Water solubility can be on the order of a few parts per million. The nominal boiling point of each fluid can be determined during their manufacture; for example, Fluorinert liquids (3M) are available with boiling points ranging from 30° C. to 215° C., and pour points as low as −101° C.

In other examples, the fluorinated liquid can be Fomblin™, Novec, Galden or any fluid listed in Table 1. The vapor parameters in Table 1 are provided at 25° C. unless otherwise noted.

TABLE 1
Fluorinated fluids and properties.

| | | | Specific | | | | |
|---|---|---|---|---|---|---|---|
| | | | Heat | Thermal | Boiling | Vapor | |
| | Viscosity | Density | (kJ/Kg | Conductivity | Point | pressure | |
| Liquid | (cSt) | (g/ml) | K) | (W m⁻¹ K⁻¹) | ° C. | (Pa) | MW |
| Fluorinert FC- | 0.71 | 1.770 | 1.10 | | 103 | 4150.0 | |
| 3255 | | | | | | | |
| Fluorinert FC-77 | 0.72 | 1.780 | 1.10 | 0.0630 | 97 | 5620.0 | 416 |
| Fluorinert FC- | 0.75 | 1.820 | 1.10 | 0.0660 | 128 | 1440.0 | 521 |
| 3283 | | | | | | | |
| HFE-7500 | 0.77 | 1.610 | 1.13 | 0.0650 | 130 | 2100.0 | 414 |
| ZT130 | 0.89 | 1.65 | 1.213 | 0.0920 | 130 | 1055.9 | 497 |
| HT110 | 0.83 | 1.72 | 0.962 | 0.0700 | 110 | 2266.5 | 580 |
| HT135 | 1 | 1.73 | 0.962 | 0.0700 | 135 | 1066.6 | 610 |
| ZT150 | 1.2 | 1.67 | 1.172 | 0.0900 | 150 | 733.3 | 572 |
| HFE-7600 | 1.07 | 1.540 | 1.32 | 0.0855 | 131 | | 346 |
| Fluorinert FC-40 | 1.80 | 1.850 | 1.10 | 0.0650 | 155 | 432.0 | 650 |
| HT170 | 1.8 | 1.77 | 0.962 | 0.0700 | 170 | 254.6 | 760 |
| ZT180 | 1.5 | 1.69 | 1.088 | 0.0880 | 178 | 240.0 | 648 |
| HFE-7800 | 1.67 | 1.724 | 1.08 | 0.0560 | 175 | 65.9 | 542 |
| HT200 | 2.4 | 1.79 | 0.962 | 0.0700 | 200 | 133.3 | 870 |
| Fluorinert FC-43 | 2.50 | 1.860 | 1.10 | 0.0650 | 174 | 192.0 | 670 |

A liquid composition for use can have a boiling point of between 95 and 500° C. In some instances, the liquid composition has a boiling point higher than PCR reaction temperatures. In some embodiments, when a device herein is used for thermal cycling of a PCR reaction, the boiling point of the liquid is greater than 100° C. In other embodiments, the liquid composition when being filled into the reservoir is at 105° C., therefore the boiling point of the fluid is greater than 105° C. In an embodiment, the liquid composition has a boiling point within 5° C. or 130° C.

In some instances, the liquid composition has a viscosity less than 10, 5, or 2.5 cSt at 25° C. In some instances, the liquid composition has a viscosity less than 2.50 cSt at 25° C. In some instances, the liquid composition has a viscosity between about 0.10 and 10 cSt at 25° C. In some instances, the liquid composition has a viscosity between about 0.70 and 2.6 cSt at 25° C. In an embodiment, the liquid composition has a viscosity less than liquid gallium. In some instances, the liquid composition has a density greater than 0.5 g/ml or greater than 1 g/ml at 25° C. In an embodiment, the liquid composition has a density greater than 1.54 g/ml at 25° C. In some instances, the liquid composition can be selected by reducing the viscosity of the fluid while increasing the density and thermal conductivity.

The liquid composition can have a vapor pressure of less than 8000 Pa at 25° C. In some embodiments, the liquid composition has a vapor pressure of less than 1500 Pa at 25° C. In some instances, the vapor pressure of the liquid composition is between 65.9 and 5620 Pa at 25° C. In some instances, vapor pressure can be an important factor when choosing a liquid composition because the liquid will be rapidly heated and cooled, therefore it will expand and contract within a closed reservoir. In some embodiments, the expansion and contraction of a liquid composition can be somewhat accounted for by placing the internal space and liquid composition within a reservoir under a vacuum.

In some instances, the liquid composition herein has a thermal conductivity of between 0.01 and 0.1 W m⁻¹K⁻¹ when the liquid composition is not being stirred. In some instances, the thermal conductivity of the liquid composition is greater than 0.1 W m⁻¹K⁻¹ when the liquid composition is not being stirred. In some instances, the liquid composition has a thermal conductivity of between 0.0560 and 0.0920 W m⁻¹K⁻¹ when the liquid composition is not being stirred. In some embodiments, the liquid composition has a thermal conductivity not less than 0.0560 W m⁻¹K⁻¹.

In some instances, the liquid composition does not oxidize. For example, the liquid composition can remain inside a reservoir of a device for the lifetime of the device and does not need to be changed or recycled. In this way, the system can remain closed when filled with a liquid composition as described herein. In some instances, the liquid composition does not foul.

A reservoir comprising a liquid can maintain a uniform temperature throughout the block. In one embodiment this is achieved through passive forces such as convection currents or passive conduction in a liquid composition. In an alternative embodiment temperature uniformity is enhanced by actively mixing the liquid metal or thermally conductive fluid using a method such as a stirring device, a circulation system, a vibration device, or magnetohydrodynamic (MHD) force.

### C. Stirring Device

The stirring device can be located within the reservoir. The stirring device can be a paddle wheel, a stir bar, a pump, or a combination thereof. In some instances, the stirring device is driven by an electric motor. In some instances, the stirring device is driven by a magnet. In an example, the stirring device is within the reservoir and magnetically couples to a driving magnet on the outside of the reservoir.

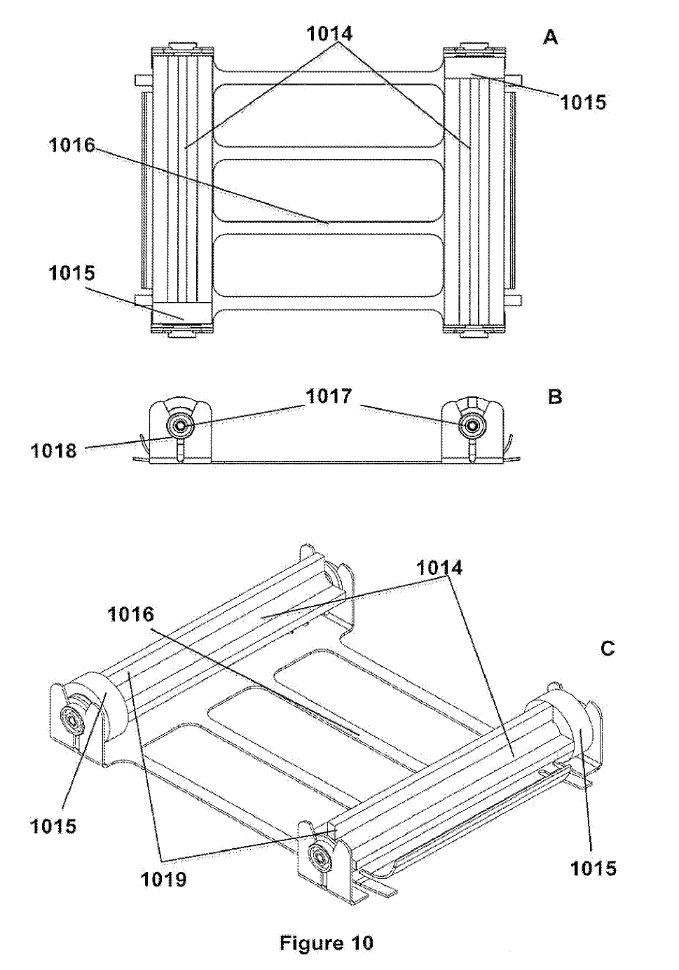

FIG. 10A illustrates exemplary stirrers 1014 of the thermal block. The two stirrers 1014 are mounted on a stirrer frame 1016 that can inserted into the reservoir of the thermal block. The stirrers 1014 comprise magnets 1015 for rotating the stirrers 1014 when couple to the device. FIG. 10B demonstrates a side view of the stirrer frame 1016 comprising notches 1018 on which the stirrers 1014 are mounted. In this example, the notches 1018 comprising bearings 1017 allow for free rotation of the stirrers 1014. FIG. 10C illustrates another exemplary view of the stirrers 1014 and stirrer frame 1016. As demonstrated in the figure, the stirrers 1014 comprise a plurality of paddles 1019 capable of moving fluid within the reservoir. In some instances, the stirrers 1014 comprise one paddle 1019. In some instances, the stirrers 1014 comprise two or more paddles 1019. The figure also demonstrates that the stirrers 1014 in some embodiment comprise a magnet 1015. The magnets 1015 can be positioned on either end of the stirrer 1014. In some instances, the stirrers 1014 comprise more than one magnet 1015.

In an embodiment the composition may be circulated by a stir bar. The stir bar may be linked to a motor which causes it to stir, or it may be magnetically responsive and stir in response to a change in magnetic field. In one embodiment the stir bar is resistant to rapid changes in temperature or it is coated with a covering that is resistant to rapid changes in temperature. In one embodiment the stir bar is a simple horizontal bar. In an alternative embodiment the stir bar may be fan shaped or have multiple projections which serve to stir the liquid composition. In yet another embodiment the liquid composition may be circulated by a vibration device. The vibration device may be integrated into an apparatus or reservoir, or it may be a secondary device. In one embodiment an acoustical device is used to vibrate the liquid composition, such as a piezo mixer, ultrasonic vibrator, subsonic vibrator or other sonic device. The vibrator may comprise speaker coils or piezos or mechanical motors.

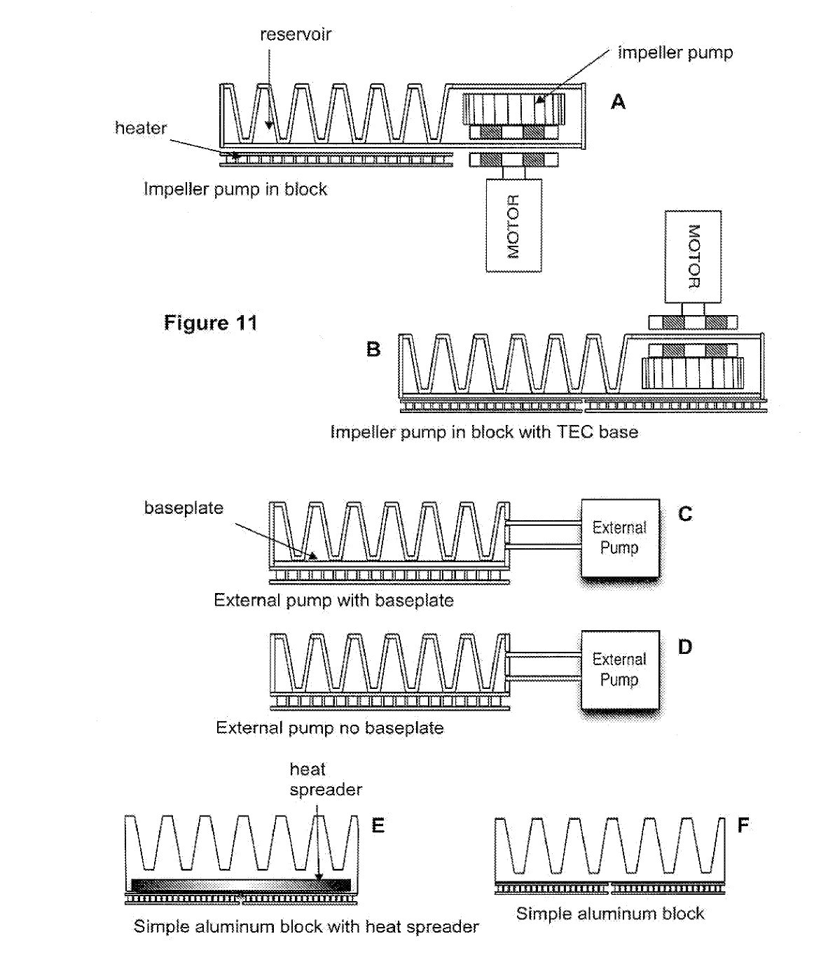

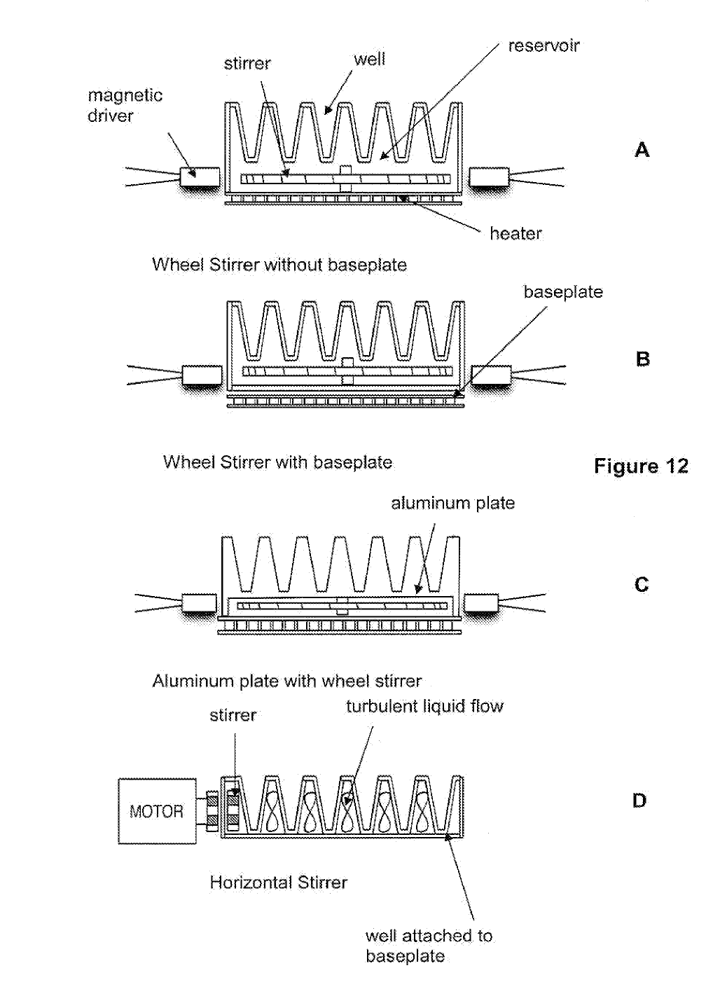

Examples of stirring devices are shown in FIGS. 11A-F and 12A-D. The figures also demonstrate exemplary embodiments of reservoir and heaters of an apparatus herein. The stirring devices in FIGS. 11A-F and 12A-D include magnetically driven stir bars, including a horizontal stir bar on one or both sides of the reservoir. Exemplary stirring devices also include external pumps and internal impeller pumps. In some embodiments, the baseplate of the reservoir is the top surface of a heater as shown in FIGS. 11A-F and 12A-D.

In an example, the stirring device moves the liquid composition by splashing the composition within the reservoir. Also, the stirring device can move the liquid composition by generating turbulent flow. In an embodiment, a stirring device moves a liquid composition at a high velocity. In some instances, the stirring device creates turbulent flow within the reservoir. In other instances, the stirring device can move the liquid composition within the reservoir such that it splashes against the inner walls of the reservoir.

When stirring a liquid, the heat transfer coefficient increases by at least 2-fold. In some instances, the heat transfer coefficient increases by at least 10-fold when stirring the liquid. In some instances, a stirring device within the apparatus vigorously stirs the liquid composition within the device. In an example, a fluorinated fluid has a heat transfer coefficient which is significantly lower than many other liquids, such as liquid gallium. However, when the fluorinated fluid is vigorously stirred, the thermal conductivity increases such that the fluid can quickly and accurately transfer heat from the heat to a sample in the sample holder. In this way, a fluid with a heat transfer coefficient when not stirred that may not transfer heat well can transfer heat much more efficiently when stirred.

A reservoir comprising a stirring device can be larger than the heater of which it is in thermal contact. This occurs because the reservoir can act as a heat spreader, and in addition to transferring near uniform temperatures across the entire reservoir and/or the sample holder in thermal contact with the reservoir, the reservoir can spread any inefficiencies or non-uniformities of temperature from the heater.

### D. Heater

In some instances, the heater is a thermoelectric device. In other instances, the heater is a resistive device. An apparatus herein can also comprise a cooler. In some instances, the heater and the cooler are the same device, for example, a Peltier device. A variety of heaters and coolers are known to a practitioner in the art. In one embodiment, a heater is a Peltier device or a resistive heater. In an embodiment, the sample block in thermal contact with a Peltier-effect thermoelectric device. In an alternative embodiment, the heater may be provided by extending a tube into the sample block through which hot or cold fluids can be pumped. In alternative embodiments, the sample block can be fitted with a heating and/or cooling coil, or with an electrical resistance heater arranged to prevent edge effects.

Peltier devices or elements, also known as thermoelectric (TE) modules, are small solid-state devices can function as heat pumps. A typical Peltier unit is a few millimeters thick by a few millimeters to a few centimeters in a square or rectangular shape. It is a sandwich formed by two ceramic plates with an array of small Bismuth Telluride (Bi₂Te₃) cubes (“couples”) in between. When a DC current is applied heat is moved from one side of the device to the other where it can be removed by a heat sink. The “cold” side may be attached to a heat sink. If the current is reversed the device changes the direction in which the heat is moved. Peltier devices lack moving parts, do not require refrigerant, do not produce noise or vibration, are small in size, have a long life, and are capable of precision temperature control. Temperature control may be provided by using a temperature sensor feedback (such as a thermistor or a solid-state sensor) and a closed-loop control circuit, which may be based on a general purpose programmable computer.

In some instances, a Peltier element of an apparatus herein is a diced Peltier. In some instances, a Peltier element of an apparatus herein is a diced Peltier. In some instances, an apparatus comprises more than one Peltier element. In some instances, an apparatus comprises a Peltier element a Kapton surface. In some instances, an apparatus comprises a Peltier element a Kapton surface. In some instances, an apparatus comprises a modified Peltier element. In some instances, an apparatus comprises more than one Peltier element.

In another embodiment the thermal cycler may further comprise an electric resistance heater and a Peltier element used in combination to obtain the required speed of the temperature changes in the sample block and the required precision and homogeneity of the temperature distribution.

A heater as described herein may also comprise a heat sink as is known to one skilled in the art. In one embodiment, a heat sink is a Peltier device, a refrigerator, an evaporative cooler, a heat pipe, a heat pump, or a phase change material. In one embodiment, the heat sink is a thermoelectric device such as a Peltier device. The heat sink may also be a heat pipe, which is a sealed vacuum vessel with an inner wick which serves to transfer heat by the evaporation and condensation of a fluid. Heat pipes which are suitable for use in the invention are disclosed, for example in WO 01/51209, U.S. Pat. No. 4,950,608, and U.S. Pat. No. 4,387,762. Similarly suitable devices are produced by the company Thermacore (Lancester, USA) and sold under the trade name Therma-Base™. Additional devices for use as heat sinks are also described in U.S. Pat. No. 5,161,609 and U.S. Pat. No. 5,819,842.

In an alternative embodiment, a heater and sometimes the reservoir is designed to maintain different temperatures in different zones of the reservoir wells. This can allow different sample wells in different zones to be cycled at different temperatures simultaneously. In one embodiment the liquid metal or thermally conductive fluid heat block is a capable of maintaining a temperature gradient across 2, 3, 4, 5, 6 or more zones. In one embodiment temperature gradients in excess of 0.1° C. to 20° C. across the reservoir can be achieved. In some embodiments the heat block will contain internal baffles or insulated walls which act to separate different zones of the liquid composition from other zones. Each zone may further comprise an individual fluid stirrer. Further each zone of the heat block may comprise individual heating and/or cooling elements such as a heat conduction element (wires, tubes), thin foil type heater, Peltier elements or cooling units. In some embodiments, an apparatus comprises a plurality of reservoirs and a plurality of heaters to create temperature zones.

### E. Sample Holder

As described herein, the sample holder can be a multiwell plate. In some instances, the multiwell plate has 16, 24, 48, 96, 384 or more sample wells. In some instances, the multiwell plate is a standard microwell plate for biological analysis. For example, the multiwell plate can be plate used for PCR. In an embodiment, the multiwell plate consists of 48 sample wells. The apparatus described herein can function to keep the temperature of the samples within each of the sample wells of a multiwell plate within ±0.3° C., ±0.2° C., or ±0.1° C. In other embodiments, the sample holder can be sample tubes, such as Eppendorf tubes. In an embodiment, the temperature variance of the device during the annealing or denaturation step of a PCR process is ±0.5° C., ±0.4° C., ±0.3° C., ±0.2° C., ±0.1° C., ±0.05° C., or ±0.01° C. or less. In an embodiment, the temperature variance of the device during the annealing or denaturation step of a PCR process is ±0.5° C., ±0.4° C., ±0.3° C., ±0.2° C., ±0.1° C., ±0.05° C., or ±0.01° C. within 30, 20, 10, 5, 3, 2, 1, or 0.5 s after changing the temperature by more than 5, 10, 20, 30, 40, or 50° C. In an embodiment, the temperature variance of the device is less than ±0.1° C. during the annealing or denaturation step of PCR.

As described herein, a sample holder can be reaction vessels of a variety of shapes and configurations. In an embodiment sample holder can be used to contain reaction mixtures, such as PCR reaction mixtures, reverse transcription reaction mixtures, real-time PCR reaction mixtures, or any other reaction mixture which requires heating, cooling or a stable uniform temperature. In one embodiment the sample holder is round or tubular shaped vessels. In an alternative embodiment the sample holder is oval vessels. In another embodiment the sample holder is rectangular or square shaped vessels. Any of the preceding embodiments may further employ a tapered, rounded or flat bottom. In yet another embodiment the sample holder is capillary tubes, such as clear glass capillary tubes or coated capillary tubes, wherein the coating (for example metal) increases internal reflectivity. In an additional embodiment the sample holder is slides, such as glass slides. In another embodiment the sample holder is sealed at the bottom. In another embodiment the sample holder is coated, at least internally, with a material for preventing an amplicon from sticking to the sample holder walls, such as a fluorinated polymer or BSA.

In one embodiment the sample holder is manufactured and used as individual vessels. In another embodiment the sample holder is a plurality of vessels linked together in a horizontal series comprising a multiple of individual vessels, such as 2, 4, 6, 10, 12, 14 or 16 tubes. In yet another embodiment the sample holder is linked together to form a sheet, plate or tray of vessels designed to fit into the top of the heating block of a thermal cycler so as to occupy some or all available reaction wells. In one embodiment the holder is a microplate comprising at least 6, wells, 12 wells, 24 wells, 36 wells, 48 wells, 54 wells, 60 wells, 66 wells, 72 wells, 78 wells, 84 wells, 90 wells or 96 wells, 144 wells, 192 wells, 384 wells, 768 wells, 1536 wells, or more wells.

In one embodiment the sample holder has caps or a cover attached to the open ends of sample wells or vessels. In one embodiment the sample wells or vessels are designed to hold a maximum sample volume, such as 10 ul, 20 ul, 30 ul, 40 ul, 50 ul, 60 ul, 70 ul, 80 ul, 90 ul, 100 ul, 200 ul, 250 ul, 500 ul, 750 ul, 1000 ul, 1500 ul, 2000 ul, 5 mL, or 10 mL. In an embodiment, the sample holder comprises polypropylene.

In some embodiments real-time polymerase chain reactions (PCR) are performed in a sample holder manufactured from materials chosen for their optical clarity and for their known non-interaction with the reactants, such as glass or plastic. In one embodiment the sample holder is designed so that light can enter and leave through the top portion of the sample wells, which may be covered with a material at least partially transparent to light. In one embodiment the sample holder is designed so that light is directed to exit through a single surface, such as the top or bottom.

In other embodiments the sample holder is manufactured from materials that are substantially internally reflective, such as reflective plastic, coated plastic (such as with metal or other reflective substances), coated glass (such as with metal or other reflective substances), doped glass (manufactured with the addition of molecules that increase the reflectivity of the glass), or metal, including but not limited to stainless steel, chromium, or other substantially non-reactive metals.

### F. Optical Assembly

In some instances, an apparatus as described herein can further comprise an optical assembly having a light source and an optical detector, wherein the optical assembly is positioned such that light from the light source is directed into the sample holder, and light from the sample holder is detected by the detector. The optical assembly can comprise a PIN photodiode, a CCD imager, a CMOS imager, a line scanner, a photodiode, a phototransistor, a photomultiplier or an avalanche photodiode. In some instances, the light source comprises one or more LEDs, laser diodes, vertical cavity surface emitting lasers (VCSELs), vertical external cavity surface emitting lasers (VECSELs), or diode pumped solid state (DPSS) lasers.

An optical detector as described herein can comprise a plurality of optical detectors, wherein at least one optical detector corresponds to a sample well in a sample microplate.

In some embodiments, an exemplary excitation optical path of the optical system comprises two LED arrays mounted on backplates. In an embodiment, the LED arrays emit the same color or wavelength of excitation energy. In another embodiment, the LED array emits a different color or wavelength of excitation energy, for example, one array emits blue excitation energy and the other array emits green excitation energy. The excitation energy from the LED array travels through a lens array. In an embodiment, the lens array is a lenslet array, comprising a lenslet corresponding to each LED, for example, 48 lenslets for 48 LEDs. After travelling through the lens array, the excitation optical path travels through excitation optics which can include without limitation filters, lens, or fiber optics. In some embodiments, the excitation energy is directed by the multifunction mirror towards the thermal block. In some embodiments, the multifunction mirror has at least two faces in the excitation path, each face corresponding to an LED array. A Fresnel lens or other optical device can be mounted above a sample holder. In an embodiment, the optical assembly comprises two fixed LED systems and four emission filters to support standard dyes, including without limition SYBR Green I, FAM, HEX, ROX, and Cy5.

In embodiment, an exemplary embodiment of the detection optical path of the optical system has emission energy emitted from the sample in the sample wells of the sample plate, for example, by fluorescence. The emission energy travels through a Fresnel lens and to the multifunction mirror as described herein. In an embodiment, the multifunction mirror is the same multifunction mirror as the multifunction mirror in the excitation optical path. In some embodiments, the multifunction mirror is a three-sided mirror to allow both the excitation optical path and detection optical path to be in the same plane in the optical assembly. In some instances, the optical assembly comprises a multifunction mirror that directs excitation energy to the sample plate from the at least one excitation optical path and that directs emission energy from the sample plate to the detection optical path.

In an embodiment, the multifunction mirror is a different mirror than that in the excitation optical path. In some embodiments, the detection optical path travels through the detecting optics which can include without limitation lenses, fiber optics, and optical filters. The optical path can then optionally travel through an optical filter. In some embodiments, the optical filter is a single longitudinal device with multiple filters that can be moved in the path to filter different wavelengths of energy. For example, depending on the wavelength of the detection dye used in the sample plate, the optical filter can be changed to filter out any excess noise not in the color range of the dye. The detection optical path ends at the detector, where the emission energy from the sample plate can be detected to complete an assay with the system as described herein.

An apparatus herein can also further comprise a control assembly which controls the apparatus, the light source, and the detector. In some instances, the control assembly comprises a programmable computer programmed to automatically process samples, run multiple temperature cycles, obtain measurements, digitize measurements into data and convert data into charts or graphs.

In various embodiments a control assembly is operatively linked to an apparatus or thermal cycler of the invention. Such a control assembly, for example, comprises a programmable computer comprising computer executable logic that functions to operate any aspect of the devices, methods and/or systems of the invention. For example, the control assembly can turn on/off or actuate motors, fans, regulating circuits, stir bars, continuous flow devices and optical assemblies. The control assembly can be programmed to automatically process samples, run multiple PCR cycles, obtain measurements, digitize measurements into data, convert data into charts/graphs and report.

Computers for controlling instrumentation, recording signals, processing and analyzing signals or data can be any of a personal computer (PC), digital computers, a microprocessor based computer, a portable computer, or other type of processing device. Generally, a computer comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of Unix, or of Linux.

In some embodiments, the control assembly executes the necessary programs to digitize the signals detected and measured from reaction vessels and process the data into a readable form (for example, table, chart, grid, graph or other output known in the art). Such a form can be displayed or recorded electronically or provided in a paper format.

In some embodiments, the control assembly controls regulating circuits linked to the thermal elements so as to regulate/control cycles temperatures of an apparatus as described herein.

In further embodiments, for example in real-time PCR, the control assembly generates the sampling strobes of the optical assembly, the rate of which is programmed to run automatically. Of course it will be apparent that such timing is adjustable for shining a light sources and operating a detector to detect and measure signals (for example, fluorescence).

In another embodiment an apparatus comprising a control assembly further comprises a means for moving sample vessels into apertures, such as wells in the receptacle of a heat block comprising a liquid composition. In an embodiment said means could be a robotic system comprising motors, pulleys, clamps and other structures necessary for moving sample vessels.

In some aspects of the invention, the devices/systems of the invention are operatively linked to a robotics sample preparation and/or sample processing unit. For example, a control assembly can provide a program to operate automated collection of samples, adding of reagents to collection tubes, processing/extracting nucleic acids from said tubes, optionally transferring samples to new tubes, adding necessary reagents for a subsequent reaction (for example, PCR or sequencing), and transferring samples to a thermal cycler according to the invention.

In some aspects, a system comprises: a thermal cycler comprising an internet connection; and a computer in communication with the thermal cycler. In some embodiments, the computer is a control system for the thermal cycler. In some instances, the computer provides instruction to the thermal cycler for controlling a heater, a temperature sensor, a heat sink, and/or a stirring motor. In some instances, an apparatus or thermal cycler herein comprises an internet connection. The internet connection can be a wireless connection, an ethernet connection, a USB connection, a firewire connection, a modem connection, or any other internet connection as would be obvious to those skilled in the art. In some embodiments, the computer is in communication with the thermal cycler through the internet connection of the thermal cycler. In some embodiments, the computer is directly coupled to the thermal cycler.

### II. Methods

In an aspect, a method of heating a biological sample comprises: positioning a sample holder containing a biological sample into thermal contact with an apparatus as described herein; and heating the biological sample contained by the sample holder with the apparatus.

In an embodiment, the method comprises performing PCR on the biological sample. The heating can comprises thermally cycling the biological sample between about 50-65° C. and about 90 to 100° C. PCR processes and methods are discussed in further detail herein.

In some instances, an apparatus herein maintains the temperature of a plurality of biological samples when heating. For example, a plurality of biological samples can be heated to 95° C. from 60° C., and within 10 s, each of the biological samples is maintained within ±0.3° C. of each other. In an embodiment, a plurality of biological samples is maintained within ±0.5° C., ±0.4° C., ±0.3° C., ±0.2° C., ±0.1° C., ±0.05° C., or ±0.01° C. of each other. In an embodiment, a plurality of biological samples are brought to a temperature within ±0.5° C., ±0.4° C., ±0.3° C., ±0.2° C., ±0.1° C., ±0.05° C., or ±0.01° C. within 30, 20, 10, 5, 3, 2, 1, or 0.5 s after changing the temperature of the biological samples by more than 5, 10, 20, 30, 40, or 50° C. When changing temperature of biological samples, for example, thermal cycling, temperature uniformity of a plurality of biological samples can be important for improving the quality of any assay or reaction products.

A method as described herein can comprise stirring a liquid composition within a reservoir. As discussed herein, stirring a liquid composition can increase the thermal conductivity of the liquid composition. In some instances, stirring a liquid composition within a reservoir can spread or distribute any heat within the reservoir. An apparatus with a reservoir or heat block that comprises a liquid composition can comprise a stirring device, and the stirring device can improve the temperature uniformity of the reservoir and the apparatus:

As described, the sample holder can be a multiwell plate and the wells of the multiwell plate contain the biological sample, wherein the biological sample is a polynucleotide sample.

In some instances, a method herein comprises providing reagents for carrying out PCR, and dyes for detecting the level of amplification to the wells containing the biological sample, thereby creating a reaction mixture.

Heating can comprise cycling the temperature of reaction mixture in the wells to perform multiple amplification cycles. In some instances, each of the amplification cycles comprise an annealing temperature and a denaturing temperature, and wherein the annealing (or denaturing or both) temperature of each amplification cycle varies by less than ±0.3° C. In some embodiments the uniformity of temperature of the liquid composition and reservoir is regulated by a step of a method herein of circulating the liquid composition in the reservoir. Circulation of the liquid metal or thermally conductive fluid can be created by natural convection or forced convection, such as by the intervention of a device including but not limited to a stir bar and a pump.

In some embodiments a method herein provides a thermal cycling ramp rate at a rate substantially faster than conventional metal heat blocks, such as at a rate of at least 5-50.5° C. per second, including but not limited to a range of at least 10-40° C. per second. In a related embodiment a method and apparatus herein can change temperature at a rate substantially faster than conventional metal heat blocks while maintaining a more uniform temperature across the heat block and/or within a sample within said heat block. In one embodiment the temperature of the biological samples in thermal contact with the heat block can be measured with glass bead thermistors (Betatherm). In another embodiment an infrared camera is used to measure the temperature of the samples. In another embodiment the temperature of the liquid sample is measured with an external probe.

In some instances, a method comprises thermally cycling a biological sample. In some instances, the thermal cycling of a biological sample can occur faster than many current standard thermal cycling devices. In an embodiment, an apparatus described herein comprising a reservoir and a stirring device can heat a PCR reaction from the annealing temperature to the denaturing temperature of the reaction in less than 10, 5, 4, 3, 2, 1, 0.5, 0.2, 0.1, or 0.05 s. In an embodiment, an apparatus described herein comprising a reservoir and a stirring device can cool a PCR reaction from the denaturing temperature to the annealing temperature of the reaction in less than 10, 5, 4, 3, 2, 1, 0.5, 0.2, 0.1, or 0.05 s.

A method herein can also further comprise optically measuring the dyes between or during each of a plurality of amplification cycles to determine the level of amplification.

In an aspect, a method of heating a biological sample as disclosed herein comprises: positioning a sample holder into thermal contact with a heater, wherein the sample holder comprises at least about 16 wells containing a biological sample and is at least 1 cm in width; and heating the biological sample within the sample holder with the heater; wherein the temperature variance of the biological sample between each of the at least about 16 wells is less than ±0.3° C. In some instances, the temperature variance is less than ±0.3° C. within 10 seconds immediately after increasing or decreasing the temperature of the biological sample more than 10° C. per second. In an embodiment, the sample holder is at least 0.1, 0.5, 1, 2, 3, 4, 5, or 10 cm in width. In an embodiment, all the wells are at the same temperature at the same time.

In another embodiment, a method is disclosed for making a thermal heat block comprising: forming a heat block having a reservoir; filling the reservoir with a fluid at a temperature greater than 90° C. through an opening in the heat block; sealing the opening such that the pressure inside the reservoir is lower than ambient pressure.

In an embodiment, the reservoir is substantially completely filled with a liquid composition as described herein. In some instances, the reservoir is (5% to 99%) filled with a liquid composition. The reservoir can be filled with 2, 4, 6, 8, 10, 12, 14 or more milliliters of fluid.

The fluid can be a fluorinated fluid as described herein. The temperature of the fluid when being filled can be about 100° C. or greater. The thermal block can be metallic, for example, comprising aluminum or silver.

In an embodiment, a reservoir (or thermal block) is formed from (1) a housing having a top surface comprising a plurality of wells and comprising a side wall, and (2) a base plate, which is sealed to the housing to form the reservoir. The wells in the housing can have a bottom, and the bottoms of the wells can be connected to the base plate.

In an embodiment, the housing is made by electroforming is made from copper, silver, aluminum, or a combination thereof.

As described herein, the reservoir can have a stirring element incorporated when making.

### III. Processes and Biological Methods

An apparatus configured as a thermal cycler can be used for disease diagnosis, drug screening, genotyping individuals, phylogenetic classification, environmental surveillance, parental and forensic identification amongst other uses. Further, nucleic acids can be obtained from any source for experimentation. For example, a test sample can be biological and/or environmental samples. Biological samples may be derived from human, other animals, or plants, body fluid, solid tissue samples, tissue cultures or cells derived therefrom and the progeny thereof, sections or smears prepared from any of these sources, or any other samples suspected to contain the target nucleic acids. Exemplary biological samples are body fluids including but not limited to blood, urine, spinal fluid, cerebrospinal fluid, sinovial fluid, ammoniac fluid, semen, and saliva. Other types of biological sample may include food products and ingredients such as vegetables, dairy items, meat, meat by-products, and waste. Environmental samples are derived from environmental material including but not limited to soil, water, sewage, cosmetic, agricultural, industrial samples, air filter samples, and air conditioning samples.

An apparatus herein can be used in any protocol or experiment that requires either thermal cycling or a heat block that can accurately maintain a uniform temperature. For example said thermal cycler can be used for polymerase chain reaction (PCR), quantitative polymerase chain reaction (qPCR), nucleic acid sequencing, ligase chain polymerase chain reaction (LCR-PCR), reverse transcription PCR reaction (RT-PCR), single base extension reaction (SBE), multiplex single base extension reaction (MSBE), reverse transcription, and nucleic acid ligation.

PCR reaction conditions typically comprise either two or three step cycles. Two step cycles have a denaturation step followed by a hybridization/elongation step. Three step cycles comprise a denaturation step followed by a hybridization step during which the primer hybridizes to the strands of DNA, followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Successful PCR amplification requires high yield, high selectivity, and a controlled reaction rate at each step. Yield, selectivity, and reaction rate generally depend on the temperature, and optimal temperatures depend on the composition and length of the polynucleotide, enzymes and other components in the reaction system. In addition, different temperatures may be optimal for different steps. Optimal reaction conditions may vary, depending on the target sequence and the composition of the primer. Thermal cyclers may be programmed by selecting temperatures to be maintained, time durations for each cycle, number of cycles, rate of temperature change and the like.

Primers for amplification reactions can be designed according to known algorithms. For example, algorithms implemented in commercially available or custom software can be used to design primers for amplifying desired target sequences. Typically, primers can range from least 12 bases, more often 15, 18, or 20 bases in length but can range up to 50+ bases in length. Primers are typically designed so that all of the primers participating in a particular reaction have melting temperatures that are within at least 5° C., and more typically within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 uM to 0.5 uM.

In one embodiment, an apparatus herein may be used for PCR, either as part of a thermal cycler or as a heat block used to maintain a single temperature. In a typical PCR cycle, a sample comprising a DNA polynucleotide and a PCR reaction cocktail is denatured by treatment in a sample block at about 90-98° C. for 10-90 seconds. The denatured polynucleotide is then hybridized to oligonucleotide primers by treatment in a sample block of the invention at a temperature of about 30-65° C. for 1-2 minutes. Chain extension then occurs by the action of a DNA polymerase on the polynucleotide annealed to the oligonucleotide primer. This reaction occurs at a temperature of about 70-75° C. for 30 seconds to 5 minutes in the sample block. Any desired number of PCR cycles may be carried out depending on variables including but not limited to the amount of the initial DNA polynucleotide, the length of the desired product and primer stringency.

In another embodiment, the PCR cycle comprises denaturation of the DNA polynucleotide at a temperature of 95° C. for about 1 minute. The hybridization of the oligonucleotide to the denatured polynucleotide occurs at a temperature of about 37-65° C. for about one minute. The polymerase reaction is carried out for about one minute at about 72.degree. C. All reactions are carried out in a multiwell plate which is inserted into the wells of a receptacle in a sample block of the invention. About 30 PCR cycles are performed. The above temperature ranges and the other numbers are not intended to limit the scope of the invention. These ranges are dependant on other factors such as the type of enzyme, the type of container or plate, the type of biological sample, the size of samples, etc. One of ordinary skill in the art will recognize that the temperatures, time durations and cycle number can readily be modified as necessary.

### A. Reverse Transcription PCR

Revere transcription refers to the process by which mRNA is copied to cDNA by a reverse transcriptase (such as Moloney murine leukemia virus (MMLV) transcriptase Avian myeloblastosis virus (AMV) transcriptase or a variant thereof) composed using an oligo dT primer or a random oligomers (such as a random hexamer or octamer). In real-time PCR, a reverse transcriptase that has an endo H activity is typically used. This removes the mRNA allowing the second strand of DNA to be formed. Reverse transcription typically occurs as a single step before PCR. In one embodiment the RT reaction is performed in a sample block of the invention by incubating an RNA sample a transcriptase the necessary buffers and components for about an hour at about 37° C., followed by incubation for about 15 minutes at about 45° C. followed by incubation at about 95° C. The cDNA product is then removed and used as a template for PCR. In an alternative embodiment the RT step is followed sequentially by the PCR step, for example in a one-step PCR protocol. In this embodiment all of the reaction components are present in the sample vessel for the RT step and the PCR step. However, the DNA polymerase is blocked from activity until it is activated by an extended incubation at 95° C. for 5⁻¹0 minutes. In one embodiment the DNA polymerase is blocked from activity by the presence of a blocking antibody that is permanently inactivated during the 95° C. incubation step.

### B. Real Time PCR

In molecular biology, real-time polymerase chain reaction, also called quantitative real time polymerase chain reaction (QRT-PCR) or kinetic polymerase chain reaction, is used to simultaneously quantify and amplify a specific part of a given DNA molecule. It is used to determine whether or not a specific sequence is present in the sample; and if it is present, the number of copies in the sample. It is the real-time version of quantitative polymerase chain reaction (Q-PCR), itself a modification of polymerase chain reaction.

The procedure follows the general pattern of polymerase chain reaction, but the DNA is quantified after each round of amplification; this is the “real-time” aspect of it. In one embodiment the DNA is quantified by the use of fluorescent dyes that intercalate with double-strand DNA. In an alternative embodiment modified DNA oligonucleotide probes that fluoresce when hybridized with a complementary DNA are used to quantify the DNA.

In another embodiment real-time polymerase chain reaction is combined with reverse transcription polymerase chain reaction to quantify low abundance messenger RNA (mRNA), enabling a researcher to quantify relative gene expression at a particular time, or in a particular cell or tissue type.

In certain embodiments, the amplified products are directly visualized with detectable label such as a fluorescent DNA-binding dye. In one embodiment the amplified products are quantified using an intercalating dye, including but not limited to SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin. For example, a DNA binding dye such as SYBR Green binds all double stranded (ds)DNA and an increase in fluorescence intensity is measured, thus allowing initial concentrations to be determined. A standard PCR reaction cocktail is prepared as usual, with the addition of fluorescent dsDNA dye and added to a sample. The reaction is then run in a liquid heatblock thermal cycler, and after each cycle, the levels of fluorescence are measured with a camera. The dye fluoresces much more strongly when bound to the dsDNA (i.e. PCR product). Because the amount of the dye intercalated into the double-stranded DNA molecules is typically proportional to the amount of the amplified DNA products, one can conveniently determine the amount of the amplified products by quantifying the fluorescence of the intercalated dye using the optical systems of the present invention or other suitable instrument in the art. When referenced to a standard dilution, the dsDNA concentration in the PCR can be determined. In some embodiments the results obtained for a sequence of interest may be normalized against a stably expressed gene (“housekeeping gene”) such as actin, GAPDH, or 18 s rRNA.

In various embodiments, labels/dyes detected by systems or devices of the invention. The term “label” or “dye” refers to any substance which is capable of producing a signal that is detectable by visual or instrumental means. Various labels suitable for use in the present invention include labels which produce signals through either chemical or physical means, such as flourescent dyes, chromophores, electrochemical moieties, enzymes, radioactive moieties, phosphorescent groups, fluorescent moieties, chemiluminescent moieties, or quantum dots, or more particularly, radiolabels, fluorophore-labels, quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), probes such as Taqman probes, TaqMan Tamara probes, TaqMan MGB probes or Lion probes (Biotools), flourescent dyes such as Sybr Green I, Sybr Green II, Sybr gold, CellTracker Green, 7-AAD, ethidium homodimer I, ethidium homodimer II, ethidium homodimer III or ethidium bromide, epitope tags such as the FLAG or HA epitope, and enzyme tags such as alkaline phosphatase, horseradish peroxidase, I²-galactosidase, alkaline phosphatase, β-galactosidase, or acetylcholinesterase and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, a luminescent material such as luminol; light scattering or plasmon resonant materials such as gold or silver particles or quantum dots; or radioactive material including ¹⁴C, ¹²³I, ¹²⁴I, ¹²⁵I, ¹³¹I, Tc99m, ³⁵S or ³H; or spherical shells, and probes labeled with any other signal generating label known to those of skill in the art. For example, detectable molecules include but are not limited to fluorophores as well as others known in the art as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland.

Intercalating dyes are detected using the devices of the invention include but are note limited to phenanthridines and acridines (for example, ethidium bromide, propidium iodide, hexidium iodide, dihydroethidium, ethidium homodimer⁻¹ and −2, ethidium monoazide, and ACMA); some minor grove binders such as indoles and imidazoles (for example, Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO⁻¹, POPO-3, YOYO⁻¹, YOYO-3, TOTO⁻¹, TOTO-3, LOLO⁻¹, BOBO⁻¹, BOBO-3, PO-PRO⁻¹, PO-PRO-3, BO-PRO⁻¹, BO-PRO-3, TO-PRO⁻¹, TO-PRO-3, TO-PRO-5, JO-PRO⁻¹, LO-PRO⁻¹, YO-PRO⁻¹, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO⁻¹3, ⁻¹6, -24, -21, -23, ⁻¹2, ⁻¹1, -20, -22, ⁻¹5, ⁻¹4, -25 (green), SYTO-81, -80, -82, -83, -84, -85 (orange), SYTO-64, ⁻¹7, -59, -61, -62, -60, -63 (red). Other detectable markers include chemiluminescent and chromogenic molecules, optical or electron density markers, etc.

As noted above in certain embodiments, labels comprise semiconductor nanocrystals such as quantum dots (i.e., Qdots), described in U.S. Pat. No. 6,207,392. Qdots are commercially available from Quantum Dot Corporation. The semiconductor nanocrystals useful in the practice of the invention include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV semiconductors such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same.

In addition to various kinds of fluorescent DNA-binding dye, other luminescent labels such as sequence specific probes can be employed in the amplification reaction to facilitate the detection and quantification of the amplified product. Probe based quantitative amplification relies on the sequence-specific detection of a desired amplified product. Unlike the dye-based quantitative methods, it utilizes a luminescent, target-specific probe (for example, TaqMan® probes) resulting in increased specificity and sensitivity. Methods for performing probe-based quantitative amplification are well established in the art and are taught in U.S. Pat. No. 5,210,015.

In another embodiment fluorescent oligonucleotide probes are used to quantify the DNA. Fluorescent oligonucleotides (primers or probes) containing base-linked or terminally-linked fluors and quenchers are well-known in the art. They can be obtained, for example, from Life Technologies (Gaithersburg, Md.), Sigma-Genosys (The Woodlands, Tex.), Genset Corp. (La Jolla, Calif.), or Synthetic Genetics (San Diego, Calif.). Base-linked fluors are incorporated into the oligonucleotides by post-synthesis modification of oligonucleotides that are synthesized with reactive groups linked to bases. One of skill in the art will recognize that a large number of different fluorophores are available, including from commercial sources such as Molecular Probes, Eugene, Oreg. and other fluorophores are known to those of skill in the art. Useful fluorophores include: fluorescein, fluorescein isothiocyanate (FITC), carboxy tetrachloro fluorescein (TET), NHS-fluorescein, 5 and/or 6-carboxy fluorescein (FAM), 5-(or 6-) iodoacetamidofluorescein, 5-{[2(and 3)-5-(Acetylmercapto)-succinyl]amino} fluorescein (SAMSA-fluorescein), and other fluorescein derivatives, rhodamine, Lissamine rhodamine B sulfonyl chloride, Texas red sulfonyl chloride, 5 and/or 6 carboxy rhodamine (ROX) and other rhodamine derivatives, coumarin, 7-amino-methyl-coumarin, 7-Amino-4-methylcoumarin-3-acetic acid (AMCA), and other coumarin derivatives, BODIPY® fluorophores, Cascade Blue® fluorophores such as 8-methoxypyrene⁻¹,3,6-trisulfonic acid trisodium salt, Lucifer yellow fluorophores such as 3,6-Disulfonate-4-amino-naphthalimide, phycobiliproteins derivatives, Alexa fluor dyes (available from Molecular Probes, Eugene, Oreg.) and other fluorophores known to those of skill in the art. For a general listing of useful fluorophores, see also Hermanson, G. T., BIOCONJUGATE TECHNIQUES (Academic Press, San Diego, 1996).

Embodiments using fluorescent reporter probes produce accurate and reliable results. Sequence specific RNA or DNA based probes are used to specifically quantify the probe sequence and not all double stranded DNA. This also allows for multiplexing—assaying for several genes in the same reaction by using specific probes with different-colored labels.

In one embodiment PCR is carried out in a device of the invention configured as a thermal cycler. In an embodiment, the thermal cycler further comprises an optical assembly. In another embodiment the sample block of the thermal cycler rapidly and uniformly modulates the temperature of samples contained within sample vessels to allow detection of amplification products in real time. In another embodiment the detection is via a non-specific nucleic acid label such as an intercalating dye, wherein the signal index, or the positive fluorescence intensity signal generated by a specific amplification product is at least 3 times the fluorescence intensity generated by a PCR control sample, such as about 3.5, 4.5, 5, 5.5, 6, 6.5, 7, 7.5, 8, 8.5, 9, 9.5, 10, 10.5, or 11. In an embodiment the thermal cycler may modulate the sample temperature by more than 10° C. per second, such as 10.5° C. per second.

In one embodiment an RNA based probe with a fluorescent reporter and a quencher held in adjacent positions is used. The close proximity of the reporter to the quencher prevents its fluorescence, it is only after the breakdown of the probe that the fluorescence is detected. This process depends on the 5′ to 3′ exonuclease activity of the polymerase used in the PCR reaction cocktail.

Typically, the reaction is prepared, as usual, with the addition of the sequence specific labeled probe the reaction commences. After denaturation of the DNA the labeled probe is able to bind to its complementary sequence in the region of interest of the template DNA. When the PCR reaction is heated to the proper extension temperature by the sample block, the polymerase is activated and DNA extension proceeds. As the polymerization continues it reaches the labeled probe bound to the complementary sequence of DNA. The polymerase breaks the RNA probe into separate nucleotides, and separates the fluorescent reporter from the quencher. This results in an increase in fluorescence as detected by the optical assembly. As PCR progresses more and more of the fluorescent reporter is liberated from its quencher, resulting in a well defined geometric increase in fluorescence. This allows accurate determination of the final, and initial, quantities of DNA.

In various applications, devices of the invention can be utilized for in vitro diagnostic uses, such as detecting infectious or pathogenic agents. In one embodiment, PCR is conducted using a device of the invention to detect various such agents, which can be any pathogen including without any limitation bacteria, yeast, fungi, virus, eukaryotic parasites, etc; infectious agent including influenza virus, parainfluenza virus, adenovirus, rhinovirus, coronavirus, hepatitis viruses A, B, C, D, E, etc, HIV, enterovirus, papillomavirus, coxsackievirus, herpes simplex virus, or Epstein-Barr virus; bacteria including Mycobacterium, Streptococcus, Salmonella, Shigella, Staphylcococcus, Neisseria, Pseudomonads, Clostridium, or E. coli. It will be apparent to one of skill in the art that the PCR, sequencing reactions and related processes are readily adapted to the devices of the invention for use to detect any infectious agents.

### EXAMPLE

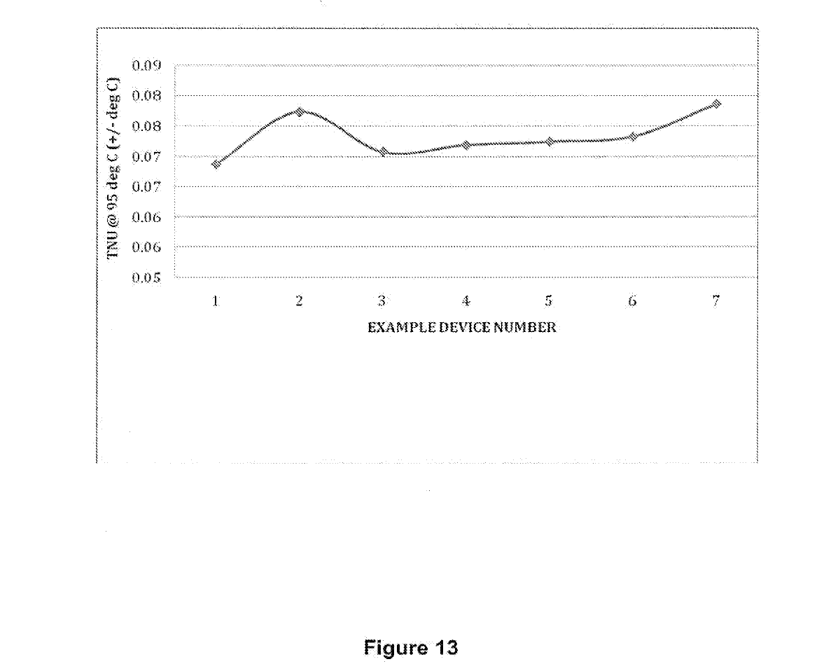

Devices and apparatuses for thermally cycling biological samples as described herein were constructed and evaluated for thermal uniformity. The devices were tested over a temperature range from 4° C. to 99° C. Temperature was measure with an array of special NIST traceable probes. 8 to 12 fitted probes were inserted into the block and measurements were made at temperatures of interest. The heated cover was held at the measurement temperature to minimize its influence on the measurement. Each temperature was measured and recorded simultaneously or near simultaneously to determine the thermal spread. FIG. 13 illustrates thermal non-uniformity (TNU) of sample devices (1-7) as described herein when the temperature of the thermal block is 95° C. The thermal non-uniformity is demonstrated as a plus/minus range. As illustrated in FIG. 12, all of the sample devices had a TNU of less than 0.09° C. at 95° C.

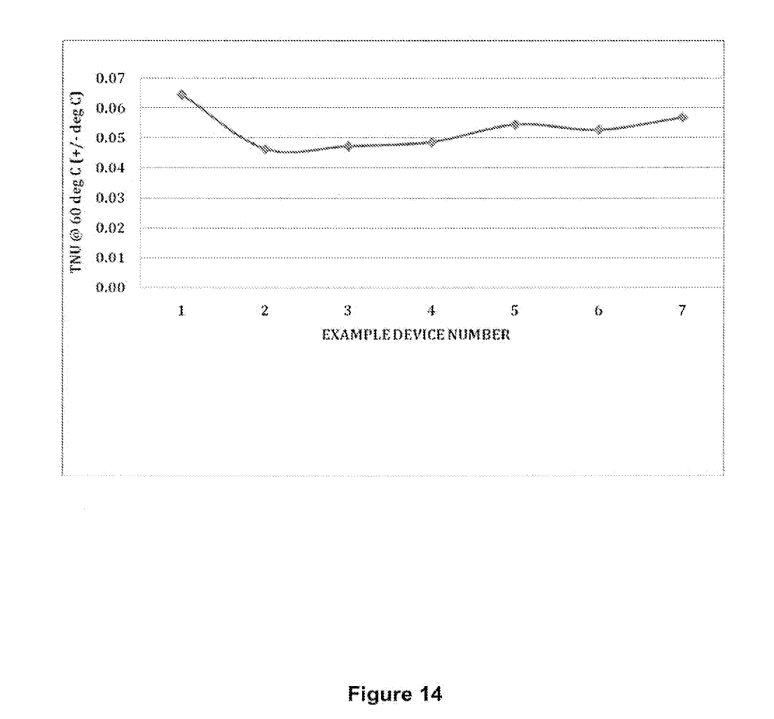

FIG. 14 illustrates thermal non-uniformity (TNU) of sample devices (1-7) as described herein when the temperature of the thermal block is 60° C. The thermal non-uniformity is, demonstrated as a plus/minus range. As illustrated in FIG. 11, all of the sample devices had a TNU of less than 0.07° C. at 60° C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus for heating a biological sample comprising:
a. a heater;
b. a reservoir in thermal contact with the heater, wherein the reservoir contains a liquid composition, wherein the liquid composition has a vapor pressure of less than about 6000 Pa at 25° C. and a thermal conductivity of greater than about 0.05 W m⁻¹K⁻¹; and
c. a stirring device configured to move the liquid composition within the reservoir,
wherein the apparatus is configured to receive a sample vessel that comprises a biological sample.

2. The apparatus of claim 1, wherein the liquid composition has a vapor pressure of less than about 1500 Pa at 25° C.

3. The apparatus of claim 1, wherein the liquid composition has a thermal conductivity of between about 0.05 and 0.1 W m⁻¹K⁻¹ when the liquid composition is not being stirred.

4. The apparatus of claim 1, wherein the liquid composition comprises a fluorinated liquid.

5. The apparatus of claim 1, wherein the liquid composition comprises Fluorinert.

6. The apparatus of claim 1, wherein the liquid composition consists essentially of a fluorinated liquid.

7. The apparatus of claim 1, wherein the liquid composition has a boiling point of between about 95 and 200° C.

8. The apparatus of claim 1, wherein the liquid composition has a viscosity less than about 2.50 cSt at 25° C.

9. The apparatus of claim 1, wherein the liquid composition has a viscosity between about 0.70 and 2.50 cSt 25° C.

10. The apparatus of claim 1, wherein the liquid composition has a vapor pressure less than water.

11. The apparatus of claim 1, wherein the reservoir is sealed.

12. An apparatus for heating a biological sample comprising: a heater, wherein the apparatus is configured to receive at least 16 sample vessels containing a biological sample, and wherein the at least 16 sample vessels are within +/−0.2° C. when heated by the heater to at least 48° C.

13. The apparatus of claim 12, wherein the apparatus is a thermal cycler and is configured to heat and cool the biological sample at PCR reaction temperatures.

14. The apparatus of claim 13, wherein the at least 16 sample vessels are within +/−0.2° C. during the PCR reaction cycles.

15. The apparatus of claim 12, wherein the heater is a thermoelectric device.

16. The apparatus of claim 15, wherein the at least 16 sample vessels are wells of a multiwell plate.

17. The apparatus of claim 16, wherein the multiwell plate has 16, 24, 48, 96, 384 or more wells.

18. The apparatus of claim 12 further comprising a reservoir comprising a liquid composition and a stirrer.

19. The apparatus of claim 18, wherein the reservoir comprises wells configured to receive the sample vessel.

20. The apparatus of claim 19, wherein the wells are anchored to a bottom surface of the reservoir.

21. The apparatus of claim 18, wherein the width by length of the heater is less than that of the reservoir.

22. The apparatus of claim 12 further comprising an optical assembly having a light source and an optical detector, wherein the optical assembly is positioned such that light from the light source is directed into the at least 16 sample vessels, and light from the at least 16 sample vessels is detected by the detector.

23. The apparatus of claim 12, wherein the optical assembly comprises a plurality of light sources, wherein each of the plurality of light sources correspond to an individual sample vessel of the at least 16 sample vessels.

24. The apparatus of claim 23, wherein the optical assembly comprises a lenslet array, wherein each lenslet corresponds to each of the plurality of light sources, to direct an excitation energy to the individual sample vessels of the at least 16 sample vessels.

25. The apparatus of claim 22, wherein the optical assembly further comprises a multifunction mirror that directs excitation energy to the at least 16 sample vessels, and wherein the multifunction mirror directs emission energy from the at least 16 sample vessels to the optical detector.

26. The apparatus of claim 22 further comprising a control assembly which controls the apparatus, the light source, and the detector.

27. The apparatus of claim 26, wherein the control assembly comprises a programmable computer programmed to automatically process samples, run multiple temperature cycles, obtain measurements, digitize measurements into data or convert data into charts or graphs.

28. The apparatus of claim 27, wherein the programmable computer is in communication with the apparatus, the light source, and the detector via an internet connection.

29. The apparatus of claim 27, wherein the programmable computer is in communication with the apparatus, the light source, and the detector via a wireless communication.

30. An apparatus for heating a biological sample comprising:
a. a heater; and
b. a reservoir in thermal contact with the heater, wherein the reservoir contains a liquid composition,
wherein the reservoir is configured to receive at least 16 sample vessels containing a biological sample, and wherein, the at least 16 sample vessels are within +/−0.2° C. when heated by the heater to at least 48° C.

31. The apparatus of claim 30, wherein the reservoir is sealed.

32. The apparatus of claim 30, wherein the liquid composition is stirred within the reservoir.

33. The apparatus of claim 30, wherein the liquid composition fluorinated fluid.

34. The apparatus of claim 30 further comprising a stirring device configured to move the liquid composition within the reservoir.

35. The apparatus of claim 34, wherein the stirring device is a paddle wheel.

36. The apparatus of claim 34, wherein the stirring device is a stir bar.

37. The apparatus of claim 34, wherein the stirring device is driven by a magnetic motor.

38. The apparatus of claim 30 further comprising an optical assembly having a light source and an optical detector, wherein the optical assembly is positioned such that light from the light source is directed into the at least 16 sample vessels, and light from the at least 16 sample vessels is detected by the detector.

39. The apparatus of claim 38, wherein the optical assembly comprises a plurality of light sources, wherein each of the plurality of light sources correspond to an individual sample vessel of the at least 16 sample vessels.

40. The apparatus of claim 39, wherein the optical assembly comprises a lenslet array, wherein each lenslet corresponds to each of the plurality of light sources, to direct an excitation energy to the individual sample vessels of the at least 16 sample vessels.

41. The apparatus of claim 38, wherein the optical assembly further comprises a multifunction mirror that directs excitation energy to the at least 16 sample vessels, and wherein the multifunction mirror directs emission energy from the at least 16 sample vessels to the optical detector.

42. An apparatus for heating a biological sample comprising:
a. a heater;
b. a reservoir in thermal contact with the heater, wherein the reservoir contains a liquid composition, wherein the liquid composition is a fluid that does not degrade within about 5 years if the reservoir is closed; and
c. a stirring device configured to move the liquid composition within the reservoir,
wherein the apparatus is configured to receive a sample vessel that comprises a biological sample.

43. The apparatus of claim 42, wherein the fluid does not oxidize within about 5 years.

44. The apparatus of claim 42, wherein the fluid is not a liquid metal.

45. The apparatus of claim 42, wherein the fluid is a fluorinated liquid.

46. The apparatus of claim 42, wherein the fluid does not degrade composition of the reservoir over time.

47. The apparatus of claim 46, wherein the reservoir comprises silver.

48. The apparatus of claim 42 further comprising an optical assembly having a light source and an optical detector, wherein the optical assembly is positioned such that light from the light source is directed into the at least 16 sample vessels, and light from the at least 16 sample vessels is detected by the detector.

49. The apparatus of claim 48, wherein the optical assembly comprises a plurality of light sources, wherein each of the plurality of light sources correspond to an individual sample vessel of the at least 16 sample vessels.

50. The apparatus of claim 49, wherein the optical assembly comprises a lenslet array, wherein each lenslet corresponds to each of the plurality of light sources, to direct an excitation energy to the individual sample vessels of the at least 16 sample vessels.

51. The apparatus of claim 49, wherein the optical assembly further comprises a multifunction mirror that directs excitation energy to the at least 16 sample vessels, and wherein the multifunction mirror directs emission energy from the at least 16 sample vessels to the optical detector.

52. An apparatus for heating a biological sample comprising:
a. a heater;
b. a reservoir in thermal contact with the heater, wherein the reservoir contains a liquid composition;
c. a heat sink in thermal contact with the heater; and
d. a thermal baseplate in thermal contact with the heater, wherein the thermal baseplate transfer heat from the heater to the heat sink.

53. The apparatus of claim 52, wherein the top surface of the thermal baseplate has similar dimensions to the bottom surface of heater in order to transfer heat uniformly to the heat sink.

54. The apparatus of claim 52, wherein the thermal baseplate comprises the same material as the interface of the heat sink.

55. The apparatus of claim 52, wherein the thermal baseplate comprises features to prevent the heater from moving horizontally when pressure is applied to the heater vertically.

56. A method of heating a biological sample comprising:
a. positioning a sample holder containing a biological sample into thermal contact with an apparatus of claim 1; and
b. heating the biological sample contained by the sample holder with the apparatus.

57. The method of claim 56, wherein the method comprises performing PCR on the biological sample.

58. The method of claim 56, wherein the apparatus maintains the temperature of sample when heating within ±0.2° C.

59. The method of claim 56 further comprising stirring the liquid composition within the reservoir.

60. The method of claim 56, wherein the heating comprises thermally cycling the biological sample between about 50-65° C. and about 90 to 100° C.

61. The method of claim 60, wherein each of the thermal cycles comprise an annealing temperature and a denaturing temperature, and wherein the annealing temperature of each amplification cycle varies by less than ±0.1° C.

62. The method of claim 60, wherein each of the thermal cycles comprise an annealing temperature and a denaturing temperature, and wherein the denaturing temperature of each amplification cycle varies by less than ±0.1° C.

63. The method of claim 56, wherein the sample holder is a multiwell plate and the wells of the multiwell plate contain the biological sample, wherein the biological sample is a polynucleotide sample.

64. The method of claim 56, further comprising providing reagents for carrying out PCR, and dyes for detecting the level of amplification to the wells containing the biological sample, thereby creating a reaction mixture.

65. The method of claim 64, further comprising optically measuring the dyes between or during each of a plurality of amplification cycles to determine the level of amplification.

66. A method of heating a biological sample comprising:
a. positioning a sample holder into thermal contact with a heater, wherein the sample holder comprises at least about 16 wells containing a biological sample and is at least 1 cm in width; and
b. heating the biological sample within the sample holder with the heater, wherein the temperature variance between at least 2 samples of the at least about 16 wells is less than ±0.2° C.

67. The method of claim 66, wherein the temperature variance is less than ±0.2° C. within 10 seconds immediately after increasing or decreasing the temperature of the biological sample more than 10° C. per second.

68. A method for making a thermal heat block comprising:
a. forming a heat block having a reservoir;
b. filling the reservoir with a fluid at a first temperature of at least 90° C. through an opening in the heat block; and
c. sealing the opening when the heat block and fluid,
wherein when the heat block is at a second temperature less than that of the first temperature, the pressure inside the reservoir is lower than ambient pressure.

69. The method of claim 68, wherein the reservoir is substantially completely filled.

70. The method of claim 68, wherein the reservoir is less than 50% filled.

71. The method of claim 68, wherein the fluid is a fluorinated fluid.

72. The method of claim 68, wherein the temperature of the fluid when being filled is about 100° C. or greater.

73. The method of claim 68, wherein the thermal block is metallic.

74. The method of claim 68, wherein the thermal block comprises wells, and wherein the bottoms of the wells are connected to the bottom of the thermal block.

75. The method of claim 68, wherein the reservoir comprises a stirring element.

76. A system comprising:
a thermal cycler comprising an internet connection; and
a computer in communication with the thermal cycler.

77. The system of claim 76, wherein the computer is in communication with the thermal cycler through the internet connection.

78. The system of claim 76, wherein the computer is in communication with the thermal cycler through a wireless connection.

79. The system of claim 76, wherein the control assembly comprises a programmable computer programmed to automatically process samples, run multiple temperature cycles, obtain measurements, digitize measurements into data and convert data into charts or graphs.

80. The system of claim 76, wherein the computer comprises the control assembly.
